(19) United States
(12) Patent Application Publication
(10) Pub. No.: US 2023/0104690 A1
(43) Pub. Date: Apr. 6, 2023

(54) MATRIX METALLOPROTEINASE INHIBITORS (MMPIs)

(71) Applicant: Brandon University, Brandon, Manitoba (CA)

(72) Inventors: Vincent CHEN, Manitoba (CA); Eric BUSHNELL, Manitoba (CA); Bryan HILL, Manitoba (CA)

(21) Appl. No.: 17/998,277

(22) PCT Filed: Jan. 21, 2021
(86) PCT No.: PCT/CA2021/050063

§ 371 Date: Nov. 9, 2022

Related U.S. Application Data
(60) Provisional application No. 63/045,461, filed on Jun. 29, 2020.

(51) Int. Cl. C07K 5/062

(52) U.S. Cl. CPC C07K 5/06043

(57) ABSTRACT

Relates to a compound of Formula I a salt, hydrate, solvate, tautomer, enantiomer, racemate, diastereomer, or combination thereof. Also, relates to a compound of Formula II a salt, hydrate, solvate, tautomer, enantiomer, racemate, diastereomer, or combination thereof. The compounds can be a matrix metalloproteinase (MMP) inhibitor. The compounds can also treat a matrix metalloproteinase mediated condition such as cancer.

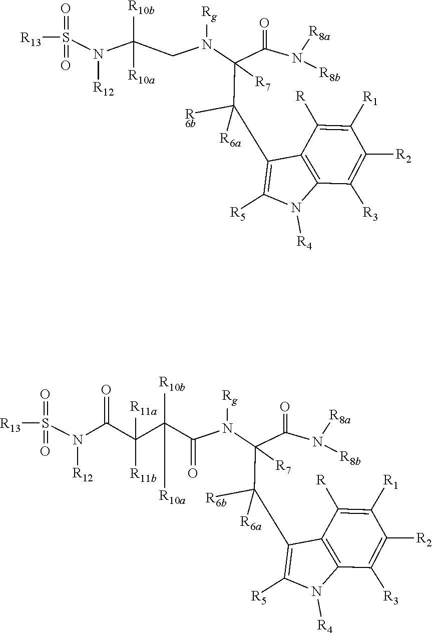

### FIELD

The present disclosure relates to matrix metalloproteinase inhibitors (MMPIs), methods of making the inhibitors, and uses thereof.

### BACKGROUND

Matrix metalloproteinases (MMPs) are a family of structurally related zinc-containing enzymes that are implicated not only in cancer progression but also inflammatory and degenerative disease processes, which are based on the breakdown of ECM. Therapeutic MMPIs have been developed to target specific MMPs linked to several types of conditions (e.g. disorders and/or diseases) such as arthritis, osteoporosis, multiple sclerosis, atherosclerosis, and carcinomas.

Glioblastoma multiforme (GBM) is a malignant tumour of the brain accounting for more than half of all astrocytoma cases (Louis, D. N et al., The 2007 WHO classification of tumours of the central nervous system (vol 114, pg 97, 2007). Acta Neuropathol. 2007, 114 (5), 547-547; and Legler, J. M. et al., Brain and other central nervous system cancers: Recent trends in incidence and mortality. JNCI-J. Natl. Cancer Inst. 1999, 91 (16), 1382-1390). High-grade gliomas are characterized by proliferation, necrosis, angiogenesis, invasion and evasion of apoptosis (Lee, J. et al., A novel NFIA-NF kappa B feed-forward loop contributes to glioblastoma cell survival. Neuro-Oncology 2017, 19 (4), 524-534; Roth, W. et al., Soluble decoy receptor 3 is expressed by malignant gliomas and suppresses CD95 ligand-induced apoptosis and chemotaxis. Cancer Research 2001, 61 (6), 2759-2765; and Furnari, F. B. et al., Malignant astrocytic glioma: genetics, biology, and paths to treatment. Genes & Development 2007, 21 (21), 2683-2710). Despite radical treatment encompassing surgical resection, radiation and chemotherapy, the GBM patients have a poor outlook. A major factor underlying the lethality of GBM, is the acquisition of therapeutic resistance and the presence of diffusely invading cells which render the complete surgical resection difficult (Rapp, M. et al., Recurrence Pattern Analysis of Primary Glioblastoma. World Neurosurg. 2017, 103, 733-740; and Sherriff, J. et al. Patterns of relapse in glioblastoma multiforme following concomitant chemoradiotherapy with temozolomide. British Journal of Radiology 2013, 86 (1022).

Infiltration of cancer is reliant on the coordination of the tumour microenvironment. In particular, the extracellular matrix (ECM) is a regulator of cancer cell invasion, migration and proliferation. Identification of genes that are differentially regulated by invasive glioma are of interest. As there is a correlation between patients outcome with the activities of proteases within the extracellular space (Fried, P. et al., Tube travel: the role of proteases in individual and collective cancer cell invasion. Cancer Res 2008, 68 (18), 7247-9), GBM processes that are mechanistically dependent upon certain proteases, such as MMPs, may be treated using MMPIs.

There is a need for MMPIs to treat MMP dependent cancers, such as GBM, and other MMP implicated conditions.

The background herein is included solely to explain the context of the disclosure. This is not to be taken as an admission that any of the material referred to was published, known, or part of the common general knowledge as of the priority date.

### SUMMARY

In accordance with an aspect, there is provided a compound of Formula I:

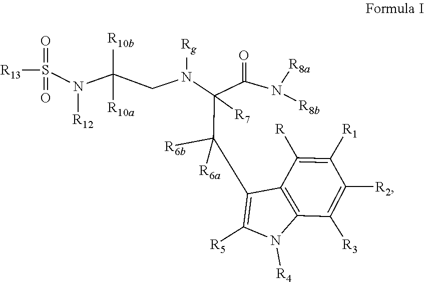

a salt, hydrate, solvate, tautomer, enantiomer, racemate, diastereomer, or combination thereof;

wherein:

R, R₁, R₂, R₃, R₄, R₅, R₆ₐ, R_{6b}, R₇, R₈ₐ, R_{8b}, R₉, R₁₀ₐ, R_{10b}, and R₁₂, are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic; and

R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

In accordance with an aspect, there is provided a compound of Formula II:

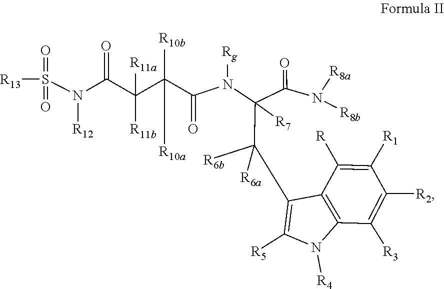

a salt, hydrate, solvate, tautomer, enantiomer, racemate, diastereomer, or combination thereof; wherein:

R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂, are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic; and

R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

With respect to aspects disclosed herein, the compound has an S configuration at the α-carbon with R₇ and an S configuration at the α-carbon with R₁₀ₐ and R_{10b}. In another aspect, wherein the compound is a matrix metalloproteinase (MMP) inhibitor. In another aspect, wherein the matrix metalloproteinase is MMP-3, MMP-8, and/or MMP-13. In another aspect,

wherein the matrix metalloproteinase is MMP-3. In another aspect, wherein R₁₃—SO₂NR₁₂— of Formula I and/or Formula II bidentately ligates to the Zn²⁺ ion of a matrix metalloproteinase (MMP) and/or is capable of forming a hydrogen bond to Glu202 of the matrix metalloproteinase (MMP).

In another aspect, there is provided a compound disclosed herein for the treatment of a matrix metalloproteinase mediated condition.

In another aspect, there is provided a pharmaceutical composition comprising the compound disclosed herein and at least one pharmaceutically acceptable carrier and/or diluent. In another aspect, the composition further comprises an anti-cancer agent.

In another aspect, there is provided, there is provided a method for the treatment of a matrix metalloproteinase mediated condition in a mammal, comprising administering to the mammal a therapeutically effective amount of the compound disclosed herein or the composition disclosed herein.

In another aspect, there is provided use of a therapeutically effective amount of the compound disclosed herein or the composition disclosed herein for the treatment of a matrix metalloproteinase mediated condition in a mammal.

With respect to aspects disclosed herein, the matrix metalloproteinase mediated condition is selected from cancer, angiogenesis, cardiovascular disease, neurological disease, inflammation, eye disease, autoimmune disease, for regulating contraception, or other conditions that are affected by the regulation of MMPs. In other aspects, wherein the cancer is selected from pancreatic cancer, gastric cancer, lung cancer, colorectal cancer, prostate cancer, cervical cancer, ovarian cancer, cancer of CNS, renal cell cancer, basal cell cancer, breast cancer, bone cancer, brain cancer, lymphoma, leukemia, melanoma, myeloma, leukemia, or other hematological cancers. In other aspects, wherein the cancer is selected from brain cancer, breast cancer, acute leukemia, chronic leukemia, colorectal cancer, or lung cancer. In other aspects, wherein the cancer is GBM. In other aspects, wherein the cancer is a carcinoma.

The novel features will become apparent to those of skill in the art upon examination of the following detailed description. It should be understood, however, that the detailed description and the specific examples presented, while indicating certain aspects of the present disclosure, are provided for illustration purposes only because various changes and modifications within the spirit and scope will become apparent to those of skill in the art from the detailed description and claims that follow.

### BRIEF DESCRIPTION OF THE DRAWINGS

The present invention will be further understood from the following description with reference to the Figures, in which:

FIG. 1: An example of a synthetic scheme for AP-1: (1) Compound A (1.0 eq.), CH₃NH₂/CH₃OH (33% CH₃NH₂ by wt., 10 eq.); (2) Compound B (1.0 eq.), FMOC-Leu-OH (2.0 eq.), DIC (2.0 eq.), Oxyma Pure (2.0 eq.), N,N-Diisopropylethylamine (2.0 eq.); (3) Compound C (1.0 eq.), 20% piperidine in DMF; and (4) Compound D (1.0 eq.), chloromethane sulfonyl chloride (7.0 eq.), N,N-Diisopropylethylamine (7.0 eq.).

FIGS. 2A-2B: Proton NMR and HPLC-MS spectra of Compound D.

FIG. 3: HPLC-MS spectra of Compound AP-1.

FIGS. 4A-4D: A) Analysis of MMP3 in C6-Cx43 (C6-13) and low motility C6 parental line confirm MMP3 expression in high motility cells. B) Relative to untreated control, zymographic assays of C6-13 conditioned media demonstrate dose-dependent loss of MMP3 activity due to ilomastat (N=3, significance level *p≤0.05, ***p≤0.001) (C). D) Examples of the Trp-Leu backbone, ilomastat and the sulfonamide-based MMPIs computationally (AP-1 to AP-7) and experimentally (AP-1 and AP-2).

FIG. 5: Example of the placement of AP-3, AP-6, AP-7 and ilomastat in the binding site of MMP3. The colour scheme is AP-3, AP-6, AP-7, and ilomastat. Regarding the molecular surface, darker regions indicate lipophilic areas and lighter regions indicates polar areas within the binding site. The Zn²⁺ ion is represented by the sphere.

FIG. 6: Examples of inhibition of MMP-3 activity detected by NFF-3 assay. Assays were monitored over six hours, relative to untreated controls, 50 μM and 100 μM concentration for each of the listed compounds were compared for: A) Ilomastat, B) Leu-Trp, and C) AP-1. Value reported here represent average, control normalized values performed in triplicate (N=3), R² values >0.99. Error bars representing standard error of the mean.

FIG. 7: Examples of the comparison of MMP3 activity (NFF-3 fluoresence, RFU/min) in the presence of Leu-Trp, AP-1, ilomastat at A) 50 μM and B) 100 μM and negative control as a function of calculated binding affinity. Anticipated inhibitory performance of (AP-3, AP-4, AP-5, AP-6 and AP-7) are based on linear regression models.

FIG. 8: Examples of another perspective of the binding of AP-3, AP-6, AP-7 and ilomastat to MMP3.

FIG. 9: Examples of a perspective of the binding of the imidazole rings of AP-6 and AP-7 to MMP3.

### DETAILED DESCRIPTION

### Definitions

Unless otherwise explained, all technical and scientific terms used herein have the same meaning as commonly understood by one of ordinary skill in the art to which this disclosure belongs. Although any methods and materials similar or equivalent to those disclosed herein can be used in the practice for testing of the present invention, the typical materials and methods are disclosed herein. The following terminology can be used.

When introducing elements disclosed herein, the articles “a”, “an”, “the”, and “said” are intended to mean that there may be one or more of the elements.

In addition, all ranges given herein include the end of the ranges and also any intermediate range points, whether explicitly stated or not. Thus, as used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. It will be understood that any embodiments described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects disclosed herein. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded from the claimed invention by way of proviso or negative limitation, such as any specific compounds or method steps, whether implicitly or explicitly defined herein.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The abbreviation, “e.g.” is derived from the Latin exempli gratia, and is used herein to indicate a non-limiting example. Thus, the abbreviation “e.g.” is synonymous with the term “for example.”

The word “or” is intended to include “and” unless the context indicates otherwise. The word “and/or” is intended to include both or either.

The phrase “at least one of” is understood to be one or more. The phrase “at least one of . . . and . . . ” is understood to mean at least one of the elements listed or a combination thereof, if not explicitly listed. For example, “at least one of A, B, and C” is understood to mean A alone or B alone or C alone or a combination of A and B or a combination of A and C or a combination of B and C or a combination of A, B, and C.

The term “therapeutically effective amount” as used herein encompasses that amount of active compound or pharmaceutical agent that elicits the biological or medicinal response in a tissue, system, animal, such as a mammal (e.g. human) that is desired. When given to treat a disorder, condition, and/or disease, it is an amount that may, when administered to a subject, including a mammal, achieve a desired result, such as treat a disease.

The compounds of the present invention may have asymmetric centers, chiral axes, and chiral planes (as described, for example, in: E. L. Eliel and S. H. Wilen, Stereo-chemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included. In addition, the compounds disclosed herein may exist as tautomers and both tautomeric forms are intended to be encompassed, even though only one tautomeric structure may be depicted. Chemical structures depicted herein, and therefore the compounds disclosed herein, encompass all of the corresponding enantiomers and stereoisomers, that is, both the stereomerically pure form (e.g., geometrically pure, enantiomerically pure, or diastereomerically pure) and enantiomeric and stereoisomeric mixtures. The term “racemic mixture” encompasses a mixture that is about 50% of one enantiomer and about 50% of the corresponding enantiomer relative to all chiral centers in the molecule. Thus, all enantiomerically-pure, enantiomerically-enriched, and racemic mixtures of the compounds disclosed herein are encompassed. Enantiomeric and stereoisomeric mixtures of compounds disclosed herein can be resolved into their component enantiomers or stereoisomers by well-known methods. Examples include the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, N Y, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Emest L. Eliel, Samuel H. Wilen and Lewis N. Manda (1994 John Wiley & Sons, Inc.), and Stereoselective Synthesis A Practical Approach, Mihaly Nogradi (1995 VCH Publishers, Inc., NY, N. Y.). Enantiomers and stereoisomers can also be obtained from stereomerically- or enantiomerically-pure intermediates, reagents, and catalysts by well-known asymmetric synthetic methods.

With respect to compound terminology, generally, reference to a certain element such as hydrogen or H is meant to, if appropriate, include all isotopes of that element.

Where the term “alkyl group” is used, either alone or within other terms such as “haloalkyl group” (e.g. halo-alkyl-) and “alkylamino group” (e.g. alkyl-NH—), “alkyl group” encompasses linear or branched carbon radicals having, for example, one to about twenty carbon atoms or, in specific embodiments, one to about twelve carbon atoms. In other embodiments, alkyl groups are “lower alkyl” groups having one to about six carbon atoms. Examples of such groups include Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (sec-Bu, sec-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₂)₂), 2-methyl-2-butyl (—C(CH₃)₂CH₂CH₃), 3-methyl-2-butyl (—CH(CH₃)CH(CH₃)₂), 3-methyl-1-butyl (CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3 dimethyl-2-butyl (—C(CH₃)₂CH(CH₃)₂), and 3,3-dimethyl-2-butyl (—CH(CH₃)C(CH₃)₃, hexyl, octyl, decyl, or dodecyl, and the like. In more specific embodiments, lower alkyl groups have one to four carbon atoms.

The term “alkenyl group” encompasses linear or branched carbon radicals having at least one carbon-carbon double bond. The term “alkenyl group” can encompass conjugated and non-conjugated carbon-carbon double bonds or combinations thereof. An alkenyl group, for example, can encompass two to about twenty carbon atoms or, in a particular embodiment, two to about twelve carbon atoms. In embodiments, alkenyl groups are “lower alkenyl” groups having two to about four carbon atoms. Examples of alkenyl groups include ethenyl, propenyl, ally, propenyl, butenyl and 4-methylbutenyl. The terms “alkenyl group” and “lower alkenyl group”, encompass groups having “cis” and “trans” orientations, or alternatively, “E” and “Z” orientations.

The term “alkynyl group” encompasses linear or branched carbon radicals having at least one carbon-carbon triple bond. The term “alkynyl group” can encompass conjugated and non-conjugated carbon-carbon triple bonds or combinations thereof. Alkynyl group, for example, can encompass two to about twenty carbon atoms or, in a particular embodiment, two to about twelve carbon atoms. In embodiments, alkynyl groups are “lower alkynyl” groups having two to about ten carbon atoms. Some examples are lower alkynyl groups having two to about four carbon atoms. Examples of such groups include propargyl, butynyl, and the like.

The term “halo” encompasses halogens such as fluorine, chlorine, bromine or iodine atoms.

The term “haloalkyl group” encompasses groups wherein any one or more of the alkyl carbon atoms is substituted with halo as defined above (e.g. halo-alkyl-). Specifically encompassed are monohaloalkyl, dihaloalkyl and polyhaloalkyl groups including perhaloalkyl. A monohaloalkyl group, for one example, may have either an iodo, bromo, chloro or fluoro atom within the group. Dihalo and polyhaloalkyl groups may have two or more of the same halo atoms or a combination of different halo groups. “Lower haloalkyl group” encompasses groups having 1-6 carbon atoms. In some embodiments, lower haloalkyl groups have one to three carbon atoms. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl group” encompasses linear or branched alkyl groups having, for example, one to about ten carbon atoms, any one of which may be substituted with one or more hydroxyl groups (e.g. HO-alkyl-). In embodiments, hydroxyalkyl groups are “lower hydroxyalkyl” groups having one to six carbon atoms and one or more hydroxyl groups. Examples of such groups include hydroxymethyl, hydroxyethyl, hydroxypropyl, hydroxybutyl and hydroxyhexyl.

The term “alkoxy group” encompasses linear or branched oxy-containing groups each having alkyl portions of, for example, one to about ten carbon atoms (e.g. alkyl-O—). In embodiments, alkoxy groups are “lower alkoxy” groups having one to six carbon atoms. Examples of such groups include methoxy, ethoxy, propoxy, butoxy and tert-butoxy. In certain embodiments, lower alkoxy groups have one to three carbon atoms. The “alkoxy” groups may be further substituted with one or more halo atoms, such as fluoro, chloro or bromo, to provide “haloalkoxy” groups. In other embodiments, lower haloalkoxy groups have one to three carbon atoms. Examples of such groups include fluoromethoxy, chloromethoxy, trifluoromethoxy, trifluoroethoxy, fluoroethoxy, and fluoropropoxy.

The term “aromatic group” or “aryl group” encompasses an aromatic group having one or more rings (e.g. Aryl-) wherein such rings may be attached together in a pendent manner or may be fused. In particular embodiments, an aromatic group is one, two or three rings. Monocyclic aromatic groups may contain 4 to 10 carbon atoms, typically 4 to 7 carbon atoms, and more typically 4 to 6 carbon atoms in the ring. Typical polycyclic aromatic groups have two or three rings. Polycyclic aromatic groups having two to three rings typically have 8 to 16 carbon atoms, preferably 8 to 14 carbon atoms in the rings. Examples of aromatic groups include phenyl, naphthyl, tetrahydronaphthyl, indanyl, biphenyl, phenanthryl, anthryl or acenaphthyl.

The term “heteroatom” encompasses an atom other than carbon. Typically, heteroatoms may be selected from sulfur, phosphorous, nitrogen and/or oxygen atoms. Groups containing more than one heteroatom may contain different heteroatoms.

The term “heteroaromatic group” or “heteroaryl group” encompasses an aromatic group having one or more rings wherein such rings may be attached together in a pendent manner or may be fused, wherein the aromatic group has at least one heteroatom (e.g. heteroaryl-). Monocyclic heteroaromatic groups may contain 4 to 10 member atoms, typically 4 to 7 member atoms, and more typically 4 to 6 member atoms in the ring. Typical polycyclic heteroaromatic groups have two or three rings. Polycyclic aromatic groups having two to three rings typically have 8 to 16 member atoms, more typically 8 to 14 member atoms in the rings. Examples of heteroaromatic groups include pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like.

The term “carbocyclic group” encompasses a saturated or unsaturated carbocyclic hydrocarbon ring. Carbocyclic groups are not aromatic. Carbocyclic groups are monocyclic or polycyclic. Polycyclic carbocyclic groups can be fused, spiro, or bridged ring systems. Monocyclic carbocyclic groups may contain 4 to 10 carbon atoms, typically 4 to 7 carbon atoms, and more typically 5 to 6 carbon atoms in the ring. Bicyclic carbocyclic groups may contain 8 to 12 carbon atoms, typically 9 to 10 carbon atoms in the rings.

The term “heterocyclic group” encompasses a saturated or unsaturated ring structure containing carbon atoms and 1 or more heteroatoms in the ring. Heterocyclic groups are not aromatic. Heterocyclic groups are monocyclic or polycyclic. Polycyclic heterocyclic groups can be fused, spiro, or bridged ring systems. Monocyclic heterocyclic groups may contain 4 to 10 member atoms (i.e., including both carbon atoms and at least 1 heteroatom), typically 4 to 7, and more typically 5 to 6 in the ring. Bicyclic heterocyclic groups may contain 8 to 18 member atoms, typically 9 or 10 member atoms in the rings. Representative heterocyclic groups include, by way of example, pyrrolidine, imidazolidine, pyrazolidine, piperidine, 1,4-dioxane, morpholine, thiomorpholine, piperazine, 3-pyrroline and the like.

The term “heterogeneous group” encompasses a saturated or unsaturated chain comprising carbon atoms and at least one heteroatom. Heterogeneous groups typically have 1 to 25 member atoms. More typically, the chain contains 1 to 12 member atoms, 1 to 10, and most typically 1 to 6. The chain may be linear or branched. Typical branched heterogeneous groups have one or two branches, more typically one branch. Typically, heterogeneous groups are saturated. Unsaturated heterogeneous groups may have one or more double bonds, one or more triple bonds, or both. Typical unsaturated heterogeneous groups have one or two double bonds or one triple bond. More typically, the unsaturated heterogeneous group has one double bond.

The term “hydrocarbon group” or “hydrocarbyl group” encompasses a chain of carbon atoms. In certain aspects, the term includes 1 to 25 carbon atoms, typically 1 to 12 carbon atoms, more typically 1 to 10 carbon atoms, and most typically 1 to 8 carbon atoms. Hydrocarbon groups may have a linear or branched chain structure. Typical hydrocarbon groups have one or two branches, typically one branch. The hydrocarbon groups encompass saturated, unsaturated, conjugated, unconjugated, and combinations thereof. Unsaturated hydrocarbon groups may have one or more double bonds, one or more triple bonds, or combinations thereof.

When the term “unsaturated” is used in conjunction with any group, the group may be fully unsaturated or partially unsaturated. However, when the term “unsaturated” is used in conjunction with a specific group defined herein, the term maintains the limitations of that specific group. For example, an unsaturated “carbocyclic group”, based on the limitations of the “carbocyclic group” as defined herein, does not encompass an aromatic group.

The terms “carboxy group” or “carboxyl group” denotes —(C═O)—O—, whether used alone or with other terms, such as “carboxyalkyl group” (e.g. alkyl-(C═O)—O—).

The term “carbonyl group” denotes —(C═O)—, whether used alone or with other terms, such as “aminocarbonyl group” (e.g. H₂N—(C═O)—).

The term “amino” encompasses the radical —NH₂ wherein one or both of the hydrogen atoms may be replaced with any suitable group such as an optionally substituted hydrocarbon group. Examples of amino groups include n-butylamino, tert-butylamino, methylpropylamino and ethyldimethylamino.

The term “cycloalkyl group” includes saturated carbocyclic groups. In certain embodiments, cycloalkyl groups include C₃-C₆ rings. In embodiments, there are compounds that include, cyclopentyl, cyclopropyl, and cyclohexyl.

The term “cycloalkenyl group” includes carbocyclic groups that have one or more carbon-carbon double bonds; conjugated or non-conjugated, or a combination thereof. “Cycloalkenyl” and “cycloalkyldienyl” compounds are included in the term “cycloalkenyl”. In certain embodiments, cycloalkenyl groups include C₃-C₆ rings. Examples include cyclopentenyl, cyclopentadienyl, cyclohexenyl and cycloheptadienyl. The “cycloalkenyl” group may have 1 to 3 substituents such as lower alkyl, hydroxyl, halo, haloalkyl, nitro, cyano, alkoxy, lower alkylamino, and the like.

The term “cycloalkylalkyl” encompasses a cycloalkyl-alkyl group wherein a cycloalkyl as described above is bonded through an alkyl, as described above. Cycloalkylalkyl groups may contain a lower alkyl moiety. Examples of cycloalkylalkyl groups include cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The terms “alkylcarbonyl group” encompasses carbonyl groups which have been substituted with an alkyl group ((e.g. alkyl-(C═O)—). In certain embodiments, “lower alkylcarbonyl group” has lower alkyl group as described above attached to a carbonyl group.

The term “aminoalkyl group” encompasses linear or branched alkyl groups having one to about ten carbon atoms any one of which may be substituted with one or more amino groups (e.g. H₂N-alkyl-). In some embodiments, the aminoalkyl groups are “lower aminoalkyl” groups having one to six carbon atoms and one or more amino groups. Examples of such groups include aminomethyl, aminoethyl, aminopropyl, aminobutyl and aminohexyl.

The term “alkylaminoalkyl group” encompasses aminoalkyl groups having the nitrogen atom independently substituted with an alkyl group (e.g. (alkyl)₂-N—). In certain embodiments, the alkylaminoalkyl groups are “loweralkylaminoalkyl” groups having alkyl groups of one to six carbon atoms. In other embodiments, the lower alkylaminoalkyl groups have alkyl groups of one to three carbon atoms. Suitable alkylaminoalkyl groups may be mono or dialkyl substituted, such as N-methylaminomethyl, N, N-dimethyl-aminoethyl, N, N-diethylaminomethyl and the like.

The term “aralkyl group” encompasses aryl-substituted alkyl groups (e.g. Aryl-alkyl-). In embodiments, the aralkyl groups are “lower aralkyl” groups having aryl groups attached to alkyl groups having one to six carbon atoms. In other embodiments, the lower aralkyl groups phenyl is attached to alkyl portions having one to three carbon atoms. Examples of such groups include benzyl, diphenylmethyl and phenylethyl. The aryl in said aralkyl may be additionally substituted with halo, alkyl, alkoxy, haloalkyl and haloalkoxy.

The term “arylalkenyl group” encompasses aryl-substituted alkenyl groups. In embodiments, the arylalkenyl groups are “lower arylalkenyl” groups having aryl groups attached to alkenyl groups having two to six carbon atoms. Examples of such groups include phenylethenyl. The aryl in said arylalkenyl may be additionally substituted with halo, alkyl, alkoxy, haloalkyl and haloalkoxy.

The term “arylalkynyl group” encompasses aryl-substituted alkynyl groups. In embodiments, arylalkynyl groups are “lower arylalkynyl” groups having aryl groups attached to alkynyl groups having two to six carbon atoms. Examples of such groups include phenylethynyl. The aryl in said aralkyl may be additionally substituted with halo, alkyl, alkoxy, haloalkyl and haloalkoxy. The terms benzyl and phenylmethyl are interchangeable.

The term “alkylthio group” encompasses groups containing a linear or branched alkyl group, of one to ten carbon atoms, attached to a divalent sulfur atom (e.g. alkyl-S—). In certain embodiments, the lower alkylthio groups have one to three carbon atoms. An example of “alkylthio” is methylthio, (CH₃S—).

The term “alkylamino group” encompasses amino groups which have been substituted with one alkyl group and with two alkyl groups, including terms “N alkylamino” and “N,N-dialkylamino” (e.g. alkyl-NH—). In embodiments, alkylamino groups are “lower alkylamino” groups having one or two alkyl groups of one to six carbon atoms, attached to a nitrogen atom. In other embodiments, lower alkylamino groups have one to three carbon atoms. Suitable “alkylamino” groups may be mono or dialkylamino such as N-methylamino, N-ethylamino, N,N-dimethylamino, N,N-diethylamino and the like.

The term “arylamino group” encompasses amino groups which have been substituted with one or two aryl groups, such as N-phenylamino (e.g. Aryl-NH—). The “arylamino” groups may be further substituted on the aryl ring portion of the group.

The term “heteroarylamino” encompasses amino groups which have been substituted with one or two heteroaryl groups, such as N-thienylamino. The “heteroarylamino” groups may be further substituted on the heteroaryl ring portion of the group.

The term “aralkylamino group” encompasses amino groups which have been substituted with one or two aralkyl groups. In other embodiments, there are phenyl-C₁-C₃-alkylamino groups, such as N-benzylamino. The “aralkylamino” groups may be further substituted on the aryl ring portion of the group.

The term “alkylaminoalkylamino group” encompasses alkylamino groups which have been substituted with one or two alkylamino groups. In embodiments, there are C₁-C₃-alkylamino-C₁-C₃-alkylamino groups.

The term “aryloxy group” encompasses optionally substituted aryl groups, as defined above, attached to an oxygen atom. Examples of such groups include phenoxy.

The term “aralkoxy group” encompasses oxy-containing aralkyl groups attached through an oxygen atom to other groups (e.g. Aryl-alkyl-O—). In certain embodiments, aralkoxy groups are “lower aralkoxy” groups having optionally substituted phenyl groups attached to lower alkoxy group as described above.

The term “thiol”, alone or in combination, refers to an —SH group.

The terms “thia”, “thio” or “sulfanyl” as used herein, alone or in combination, refer to a —S— group or an ether wherein the oxygen is replaced with sulfur. The oxidized derivatives of the thio group are referred to as sulfinyl or thionyl (—S(O)—) and sulfonyl (—SO₂—). Other groups include, for example, the groups disclosed herein substituted with sulfanyl, sulfinyl and/or sulfonyl groups or sulfanyl, sulfinyl and/or sulfonyl groups substituted with the groups disclosed herein. Examples include the group —SR²⁰ wherein R²⁰ is selected from any suitable group herein, including for example: C₁-C₈ alkyl, C₃-C₁₀ cycloalkyl, 4-10 membered heterocycloalkyl, C₆-C₁₀ aryl, aralkyl, 5-10 membered heteroaryl, and heteroaralkyl; C₁-C₈alkyl substituted with halo, substituted or unsubstituted amino, or hydroxy; C₃-C₁₀ cycloalkyl, 4-10 membered heterocycloalkyl, C₆-C₁₀ aryl, aralkyl, 5-10 membered heteroaryl, or heteroaralkyl, each of which is substituted by unsubstituted C₁-C₄alkyl, halo, unsubstituted C₁-C₄ alkoxy, unsubstituted C₁-C₄ haloalkyl, unsubstituted C1-C₄ hydroxyalkyl, or unsubstituted C₁-C₄ haloalkoxy or hydroxy. Other examples include —S—(C₁-C₈ alkyl), —S—(C₃-C₁₀ cycloalkyl), —S—(CH₂)ₜ(C₆-C₁₀ aryl), —S —(CH₂)ₜ(5-10 membered heteroaryl), —S—(CH₂)ₜ(C₃-C₁₀cycloalkyl), and —S—(CH₂)ₜ(4-10 membered heterocycloalkyl), wherein t is an integer from 0 to 4 and any aryl, heteroaryl, cycloalkyl or heterocycloalkyl groups present, may themselves be substituted by unsubstituted C₁-C₄ alkyl, halo, unsubstituted C₁-C₄alkoxy, unsubstituted C₁-C₄ haloalkyl, unsubstituted C₁-C₄ hydroxyalkyl, or unsubstituted C₁-C₄ haloalkoxy or hydroxy. The term ‘sulfanyl’ includes the groups ‘alkylsulfanyl’ or ‘alkylthio’, ‘alkylthio’ or ‘alkylsulfanyl’, ‘cycloalkylsulfanyl’ or ‘cycloalkylthio’, ‘cycloalkylsulfanyl’ or ‘cycloalkylthio’, ‘arylsulfanyl’ or ‘arylthio’ and ‘heteroarylsulfanyl’ or ‘heteroarylthio’.

The terms “alkylthio” or “alkylsulfanyl” encompasses —S-alkyl where alkyl is any alkyl as defined herein. For example, the alkyl can be a C₁-C₈ alkyl. Examples include methylthio, ethylthio, propylthio and butylthio.

The terms “thioalkyl” or “sulfanylalkyl” encompasses (HS-alkyl-) where alkyl is any alkyl as defined herein. For example, the alkyl can be a C₁-C₈ alkyl. Examples include thiomethyl, thioethyl, thiopropyl and thiobutyl.

The term “alkylcarbonylthioalkyl” encompasses alkyl-(CO)—S-alkyl-.

The term “arylthio group” encompasses aryl groups of six to ten carbon atoms, attached to a divalent sulfur atom (e.g. Aryl-S—). An example of “arylthio” is phenylthio. The term “aralkylthio group” encompasses aralkyl groups as described above, attached to a divalent sulfur atom. In certain embodiments there are phenyl-C₁-C₃-alkylthio groups. An example of “aralkylthio” is benzylthio.

The term “suitable substituent”, “substituent” or “substituted” used in conjunction with the groups disclosed herein refers to a chemically acceptable group, i.e., a moiety that maintains the utility of compounds disclosed herein. For example, “substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced. It is understood that substituents and substitution patterns on the compounds may be selected by one of ordinary skill in the art to provide compounds that are chemically stable and that can be readily synthesized by techniques known in the art, as well as those methods set forth below. For example, if a substituent is itself substituted with more than one group, it is understood that these multiple groups may be on the same carbon/member atom or on different carbons/member atoms, as long as a stable structure results. Examples of some suitable substituents include, cycloalkyl, heterocyclyl, hydroxyalkyl, benzyl, carbonyl, halo, haloalkyl, perfluoroalkyl, perfluoroalkoxy, alkyl, alkenyl, alkynyl, hydroxy, oxo, mercapto, alkylthio, alkoxy, aryl or heteroaryl, aryloxy or heteroaryloxy, aralkyl or heteroaralkyl, aralkoxy or heteroaralkoxy, HO—(C═O)—, amido, amino, alkyl- and dialkylamino, cyano, nitro, carbamoyl, alkylcarbonyl, alkoxycarbonyl, alkylaminocarbonyl, dialkylaminocarbonyl, arylcarbonyl, aryloxycarbonyl, alkylsulfonyl, and arylsulfonyl. Typical substituents include aromatic groups, substituted aromatic groups, hydrocarbon groups including alkyl groups such as methyl groups, substituted hydrocarbon groups such as benzyl, and heterogeneous groups including alkoxy groups such as methoxy groups.

The term “fused” encompasses two adjoining rings, e.g., the rings are “fused rings” having two or more carbons/member atoms that are common to the two adjoining rings.

The pharmaceutically acceptable salts of the compounds disclosed herein include the conventional non-toxic salts of the compounds as formed, e.g., from non toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. The pharmaceutically acceptable salts of the compounds can be synthesized from the compounds of which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base. The compounds disclosed herein include pharmaceutically acceptable salts, solvates and prodrugs of the compounds and mixtures thereof.

The term “pharmaceutically acceptable” includes those compounds, materials, compositions, and/or dosage forms which are, within the scope of sound medical judgment, suitable for use in contact with the tissues of humans and animals without excessive toxicity, irritation, allergic response, or other problem or complication commensurate with a reasonable benefit/risk ratio.

The phrase “pharmaceutically acceptable carrier” encompasses media generally accepted in the art for the delivery of biologically active agents to mammals, e.g., humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Examples of a pharmaceutically acceptable carrier include hyaluronic acid and salts thereof, and microspheres (including, but not limited to poly(D,L)-lactide-co-glycolic acid copolymer (PLGA), poly(L-lactic acid) (PLA), poly(caprolactone (PCL) and bovine serum albumin (BSA)). Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985. Pharmaceutically acceptable carriers particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil. The compositions may also be formulated as suspensions including a compound of disclosed herein in admixture with at least one pharmaceutically acceptable excipient suitable for the manufacture of a suspension. In yet another embodiment, pharmaceutical compositions may be formulated as dispersible powders and granules suitable for preparation of a suspension by the addition of suitable excipients. Carriers suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan mo[pi]ooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin. Cyclodextrins may be added as aqueous solubility enhancers. Preferred cyclodextrins include hydroxypropyl, hydroxyethyl, glucosyl, maltosyl and maltotriosyl derivatives of [alpha]-, [beta]-, and [gamma]-cyclodextrin. The amount of solubility enhancer employed will depend on the amount of the compound disclosed herein in the composition.

The term “formulation” or composition can encompasse a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier.

The term “derivative” generally refers to a molecule that has been modified and/or changed in any way relative to a reference molecule or starting molecule.

The term “leaving group” is well understood in the art and is a molecular fragment that departs with a pair of electrons in a heterolytic bond cleavage. Leaving groups can be anions or neutral molecules, and is able to stabilize the additional electron density that results from bond heterolysis.

The term “protecting group” encompasses any group which, when bound to a hydroxyl, nitrogen, or other heteroatom prevents undesired reactions from occurring at this group and which can be removed by conventional chemical or enzymatic steps to re-establish the amino group. The particular removable blocking group employed is not critical and preferred removable amino blocking groups include conventional substituents that can be introduced chemically onto an amino functionality and later selectively removed, for example, by chemical methods in mild conditions compatible with the nature of the product. A large number of protecting groups and corresponding chemical cleavage reactions are described in Protective Groups in Organic Synthesis, Theodora W. Greene (John Wiley & Sons, Inc., New York, 1991, ISBN 0-471-62301-6). Included therein are nitrogen protecting groups, for example, amide-forming groups. In particular, see Chapter 1, Protecting Groups: An Overview, pages 1-20, Chapter 2, Hydroxyl Protecting Groups, pages 21-94, Chapter 4, Carboxyl Protecting Groups, pages 118-154, and Chapter 5, Carbonyl Protecting Groups, pages 155-184. See also Kocienski, Philip J.; Protecting Groups (Georg Thieme Verlag Stuttgart, New York, 1994). Typical nitrogen protecting groups described in Greene (pages 14-118) include benzyl ethers, silyl ethers, esters including sulfonic acid esters, carbonates, sulfates, and sulfonates. For example substituted methyl ethers; substituted ethyl ethers; p-chlorophenyl, p-methoxyphenyl, 2,4-dinitrophenyl, benzyl; substituted benzyl ethers (p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2- and 4-picolyl, diphenylmethyl, 5-dibenzosuberyl, triphenylmethyl, p-methoxyphenyldiphenyl methyl, di(p-methoxyphenyl)phenylmethyl, tri(p-methoxyphenyl)methyl, 1,3-benzodithiolan-2-yl, benzisothiazolyl S,S-dioxido); silyl ethers (silyloxy groups) (trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, tri-p-xylylsilyl, triphenylsilyl, diphenylmethylsilyl, t-butylmethoxyphenylsilyl); esters (formate, benzoylformate, acetate, choroacetate, dichloroacetate, trichloroacetate, trifluoro acetate, methoxyacetate, triphenylmethoxyacetate, phenoxyacetate, p-chlorophenoxyacetate, 3-phenylpropionate, 4-oxopentanoate (levulinate), pivaloate, adamantoate, crotonate, 4-methoxycrotonate, benzoate, p-phenylbenzoate, 2,4,6-trimethylbenzoate (mesitoate)); carbonates (methyl, 9-fluorenylmethyl, ethyl, 2,2,2-trichloroethyl, 2-(trimethylsilyl)ethyl, 2-(phenylsulfonyl)ethyl, 2-(triphenylphosphonio)ethyl, isobutyl, vinyl, ally, o-nitrophenyl, benzyl, p-methoxybenzyl, 3,4-dimethoxybenzyl, o-nitrobenzyl, p-nitrobenzyl, S-benzyl thiocarbonate, 4-ethoxy-1-naphthyl, methyl dithiocarbonate); groups with assisted cleavage (2-iodobenzoate, 4-azidobutyrate, 4-nitro-4-methylpentanoate, o-(dibromomethyl)benzoate, 2-formylbenzenesulfonate, 2-(methylthiomethoxy) ethyl carbonate, 4-(methylthiomethoxy)butyrate, miscellaneous esters (2,6-dichloro-4-methylphenoxyacetate, 2,6-dichloro-4-(1,1,3,3 tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate (tigloate), o-(methoxycarbonyl)benzoate, p-poly-benzoate, α-naphthoate, nitrate, alkyl N,N,N′,N′-tetramethyl-phosphorodiamidate, n-phenylcarbamate, borate, 2,4-dinitrophenylsulfenate, fluorenylmethyloxycarbonyl chloride (fmoc)); and sulfonates (sulfate, methanesulfonate (mesylate), benzylsulfonate, tosylate, triflate).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “condition” indicates, for example, a physical status of a mammal (as a whole or as one or more of its parts), that does not conform to a standard physical status associated with a state of well-being for the mammal. Conditions herein described include but are not limited to disorders and diseases wherein the term “disorder” indicates, for example, a condition of the mammal that is associated to a functional abnormality of the mammal or of any of its parts, and the term “disease” indicates, for example, a condition of the mammal that impairs normal functioning of the body of the mammal or of any of its parts and is typically manifested by distinguishing signs and symptoms. Typically, the compounds and compositions disclosed herein are useful for treating MMP mediated conditions.

The term “administration” (e.g., “administering” a compound) in reference to a compound, composition and/or formulation disclosed herein includes, for example, introducing the compound, composition and/or formulation into the system of the mammal in need of treatment. When a compound, composition and/or formulation is provided in combination with one or more other active agents, “administration” and its variants are each understood to include concurrent and sequential introduction of the compound, composition and/or formulation and other agents.

The term “treating cancer” or “treatment of cancer” refers to administration to a mammal afflicted with a cancerous condition and refers to an effect that alleviates the cancerous condition by, for example, killing the cancerous cells, but may also result in the inhibition of growth and/or metastasis of the cancer.

MMPs in cancer biology has led to the development of matrix metalloproteinase inhibitors (MMPIs). MMPs are zinc-dependent endopeptidases, that can rely upon the coordination substrate and metal ion to hydrolyze peptide bonds. The majority of MMPIs tend to utilize a hydroxamic acid ZBG (zinc binding group) to inactivate pathways dependent on this family of enzymes (Auge, F. et al., A novel strategy for designing specific gelatinase A inhibitors: potential use to control tumor progression. Crit. Rev. Oncol./Hematol. 2004, 49 (3), 277-282), including ilomastat (i.e. decreasing the invasiveness of high-grade astrocytoma via MMP3 inhibition (Mercapide, J. et al., Stromelysin-1/matrix metalloproteinase-3 (MMP-3) expression accounts for invasive properties of human astrocytoma cell lines. Int. J. Cancer 2003, 106 (5), 676-682)).

In embodiments, the MMPIs disclosed herein may improve pharmacokinetics, transition metal/MMP selectivity, and/or slower metabolism compared to the hydroxamic acid ZBGs. Alternative functional groups capable of inhibiting the activity of MMPs are disclosed herein. Certain MMPIs were selected based on the safety profiles of those commonly associated with sulfonamide-based medicines (a.k.a. “sulfa” drugs) (Scozzafava, A. et al., Carbonic anhydrase and matrix metalloproteinase inhibitors: Sulfonylated amino acid hydroxamates with MMP inhibitory properties act as efficient inhibitors of CA isozymes I, II, and IV, and N-hydroxysulfonamides inhibit both these zinc enzymes. J. Med. Chem. 2000, 43 (20), 3677-3687; and Apaydin, S. et al., Sulfonamide derivatives as multi-target agents for complex diseases. Bioorg Med Chem Lett 2019, 29 (16), 2042-2050) and the zinc-binding ability of the selected functional groups. Structure-activity relationships of sulfa-based inhibitors were determined and the inhibitory performance of certain compounds disclosed herein, based on the ilomastat (Leu-Trp) backbone, were measured in matrices related to Glioblastoma multiforme (GBM) (Locasale, J. W. et al., Phosphoglycerate dehydrogenase diverts glycolytic flux and contributes to oncogenesis. Nat Genet 2011, 43 (9), 869-74).

In embodiments and based upon the connexin43 (Cx43) model of glioma migration invasion, a computational framework to evaluate MMP inhibition in materials relevant to treating MMP mediated conditions (e.g. disorders and/or diseases), such as GBM, was developed. Using the ilomastat Leu-Try backbone, sulfonamide-based ZBG replacements disclosed herein can have broad utility as MMPIs. In embodiments, the performance of the compounds disclosed herein was monitored in conditioned media expressing MMP3.

The present disclosure relates to sulfonamide-based inhibitors, including derivatives thereof, methods of making the inhibitors, and uses thereof, including, for example, the use as inhibitors of MMPs. In more specific embodiments, the compounds disclosed herein include sulfonamide zinc-binding groups.

### Sulfonamide-Based Inhibitors

Sulfonamide-based inhibitors are disclosed herein. Certain embodiments include a compound of Formula I:

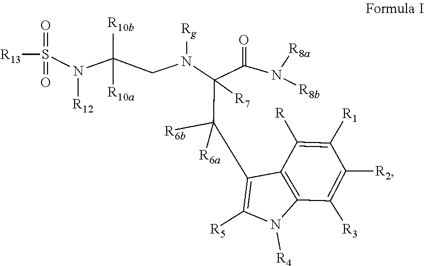

a salt, hydrate, solvate, tautomer, enantiomer, racemate, diastereomer, or combination thereof;
wherein:

R, R₁, R₂, R₃, R₄, R₅, R₆ₐ, R_{6b}, R₇, R₈ₐ, R_{8b}, R₉, R₁₀ₐ, R_{10b}, and R₁₂, are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic; and

R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

Certain embodiments of the sufonamide inhibitor include a compound of Formula II:

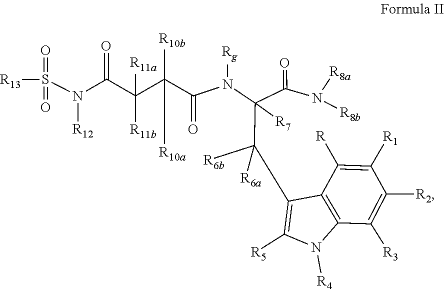

a salt, hydrate, solvate, tautomer, enantiomer, racemate, diastereomer, or combination thereof; wherein:

R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂, are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic; and

R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

In specific embodiments of Formula I and Formula II, R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, a substituted or unsubstituted alkyl group, a substituted or unsubstituted alkenyl group, a substituted or unsubstituted alkynyl group, a substituted or unsubstituted aromatic group, a substituted or unsubstituted heteroaromatic group, a substituted or unsubstituted carbocyclic group, or a substituted or unsubstituted heterocyclic group. In more particular embodiments, R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, a substituted or unsubstituted alkyl group, a substituted or unsubstituted haloalkyl group, a substituted or unsubstituted hydroxyalkyl group, a substituted or unsubstituted cyanoalkyl group, a substituted or unsubstituted alkenyl group, a substituted or unsubstituted C₁-C₆ alkylcarbonyl group, a substituted or unsubstituted alkynyl group, a substituted or unsubstituted cycloalkyl group, a substituted or unsubstituted cycloalkenyl group, a substituted or unsubstituted alkylcycloalkyl group, a substituted or unsubstituted alkylcycloalkenyl group, a substituted or unsubstituted heterocycloalkyl group, a substituted or unsubstituted alkylheterocycloalkyl group, a substituted or unsubstituted heterocycloalkenyl group, a substituted or unsubstituted alkylheterocycloalkenyl group, a substituted or unsubstituted aryl group, a substituted or unsubstituted heteroaryl group, a substituted or unsubstituted alkylaryl group, or a substituted or unsubstituted alkylheteroaryl group. In more particular embodiments, R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted alkyl group. In further embodiments, R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted C₁-C₆ alkyl group. In other embodiments, R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2 methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In other embodiments, R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3 methyl-3-pentyl, 2-methyl-3-pentyl, 2,3-dimethyl-2-butyl, 3,3-dimethyl-2-butyl, or hexyl. In more specific embodiments, R, R₁, R₂, R₃, R₄, R₅, R₈ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, or t-butyl.

In other embodiments, R, R₁, R₂, R₃, R₄, and R₅ are each H and R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted alkyl group. In further embodiments, R, R₁, R₂, R₃, R₄, and R₅ are each H and R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted C₁-C₆ alkyl group. In other embodiments, R, R₁, R₂, R₃, R₄, and R₅ are each H and R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3 dimethyl-2-butyl, 3,3-dimethyl-2-butyl, or hexyl. In other embodiments, R, R₁, R₂, R₃, R₄, and R₅ are each H and R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3 methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In more specific embodiments, R, R₁, R₂, R₃, R₄, and R₅ are each H and R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, or t-butyl.

In other embodiments, R, R₁, R₂, R₃, R₄, R₅, R₆ₐ, R_{6b}, R₇, R₉, R₁₁ₐ, R_{11b}, and R₁₂ are each H and R₈ₐ, R_{8b}, R₁₀ₐ, and R_{10b} are each independently selected from H or a substituted or unsubstituted alkyl group. In further embodiments, R, R₁, R₂, R₃, R₄, R₅, R₆ₐ, R_{6b}, R₇, R₉, R₁₁ₐ, R_{11b}, and R₁₂ are each H and R₈ₐ, R_{8b}, R₁₀ₐ, and R_{10b} are each independently selected from H or a substituted or unsubstituted C₁-C₆ alkyl group. In other embodiments, R, R₁, R₂, R₃, R₄, R₅, R₆ₐ, R_{6b}, R₇, R₉, R₁₁ₐ, R_{11b}, and R₁₂ are each H and R₈ₐ, R_{8b}, R₁₀ₐ, and R_{10b} are each independently selected from H or a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In other embodiments, R, R₁, R₂, R₃, R₄, R₅, R₆ₐ, R_{6b}, R₇, R₉, R₁₁ₐ, R_{11b}, and R₁₂ are each H and R₈ₐ, R_{8b}, R₁₀ₐ, and R_{10b} are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3 dimethyl-2-butyl, 3,3-dimethyl-2-butyl, or hexyl. In more specific embodiments, R, R₁, R₂, R₃, R₄, R₅, R₆ₐ, R_{6b}, R₇, R₉, R₁₁ₐ, R_{11b}, and R₁₂ are each H and R₈ₐ, R_{8b}, R₁₀ₐ, and R_{10b} are each independently selected from H, methyl, ethyl, 1-propyl, i-propyl, 1-butyl, i-butyl, sec-butyl, or t-butyl.

In other embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H and R_{8b} and R_{10b} are each independently selected from H or a substituted or unsubstituted alkyl group. In further embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H and R_{8b} and R_{10b} are each independently selected from H or a substituted or unsubstituted C₁-C₆ alkyl group. In other embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H and R_{8b} and R_{10b} are each independently selected from H or a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3 dimethyl-2-butyl, 3,3-dimethyl-2-butyl, or hexyl. In other embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H and R_{8b} and R_{10b} are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3 methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In more specific embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H and R_{8b} and R_{10b} are each independently selected from H, methyl, ethyl, 1-propyl, i-propyl, 1-butyl, i-butyl, sec-butyl, or t-butyl.

In more specific embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H, R_{8b} is selected from methyl or ethyl, and R_{10b} is selected from methyl, ethyl, 1-propyl, i-propyl, 1-butyl, i-butyl, sec-butyl, ort-butyl. In more specific embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H, R_{8b} is selected from methyl, and R_{10b} is selected from 1-butyl, i-butyl, sec-butyl, or t-butyl. In more specific embodiments, R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H, R_{8b} is methyl, and R_{10b} is i-butyl.

In specific embodiments of Formula I and Formula II, and in combination with any of the embodiments listed herein with respect to R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂; R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic. In other embodiments, R₁₃ is selected from H, hydroxyl group, a substituted or unsubstituted thiol group, a substituted or unsubstituted amino group, a substituted or unsubstituted alkyl group, a substituted or unsubstituted alkenyl group, a substituted or unsubstituted alkynyl group, a substituted or unsubstituted aromatic group, a substituted or unsubstituted heteroaromatic group, a substituted or unsubstituted carbocyclic group, or a substituted or unsubstituted heterocyclic group. In more particular embodiments, R₁₃ is selected from H, hydroxyl group, a substituted or unsubstituted thiol group, a substituted or unsubstituted amino group, a substituted or unsubstituted alkyl group, a substituted or unsubstituted haloalkyl group, a substituted or unsubstituted hydroxyalkyl group, a substituted or unsubstituted aminoalkyl group, a substituted or unsubstituted thioalkyl group, a substituted or unsubstituted alkylamino group, a substituted or unsubstituted alkylthio group, a substituted or unsubstituted alkoxy group, a substituted or unsubstituted carboxy group, a substituted or unsubstituted carbonyl group, a substituted or unsubstituted alkenyl group, a substituted or unsubstituted alkynyl group, a substituted or unsubstituted cyanoalkyl, a substituted or unsubstituted alkenyl, a substituted or unsubstituted C₁-C₆ alkylcarbonyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, or a substituted or unsubstituted heteroaryl.

In more particular embodiments, R₁₃ is selected from H, hydroxyl group, substituted or unsubstituted alkyl group, substituted or unsubstituted alkenyl group, substituted or unsubstituted alkynyl group, —NR₁₄R₁₅, —CR₁₆R₁₇R₁₈, —C(O)NR₁₉R₂₀, halo group, —S(O)R₂₁, —SO₂R₂₂, —R₂₃S(O)R₂₄, —R₂₅SO₂R₂₆, —R₂₇SR₂₈, —R₂₇S—C(O)R₂₈, —SR₂₉, —S—C(O)R₃₀, —C(O)SR₃₁, —N(R₃₂)C(O)R₃₃, —C(O)R₃₄, —C(O)OR₃₅, or —OR₃₆, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic. In further embodiments, R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group, a substituted or unsubstituted haloalkyl group, a substituted or unsubstituted hydroxyalkyl group, a substituted or unsubstituted cyanoalkyl group, a substituted or unsubstituted alkenyl group, a substituted or unsubstituted C₁-C₆alkylcarbonyl group, a substituted or unsubstituted alkynyl group, a substituted or unsubstituted cycloalkyl group, a substituted or unsubstituted cycloalkenyl group, a substituted or unsubstituted alkylcycloalkyl group, a substituted or unsubstituted alkylcycloalkenyl group, a substituted or unsubstituted heterocycloalkyl group, a substituted or unsubstituted alkylheterocycloalkyl group, a substituted or unsubstituted heterocycloalkenyl group, a substituted or unsubstituted alkylheterocycloalkenyl group, a substituted or unsubstituted aryl group, or a substituted or unsubstituted heteroaryl group. In more particular embodiments, R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group. In further embodiments, R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, or a substituted or unsubstituted C₁-C₆ alkyl group. In other embodiments, R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2pentyl, 4-methyl-2-pentyl, 3-methyl-3-pentyl, 2-methyl-3-pentyl, 2,3-dimethyl-2-butyl, 3,3-dimethyl-2-butyl, or hexyl. In other embodiments, R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, t-butyl, 1-pentyl, 2 pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In more specific embodiments, R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, or t-butyl.

In more particular embodiments, R₁₃ is selected from —NR₁₄R₁₅, —CR₁₆R₁₇R₁₈, —R₂₇SR₂₈, —R₂₇S—C(O)R₂₈, —C(O)SR₃₁, —N(R₃₂)C(O)R₃₃, —C(O)R₃₄, —C(O)OR₃₅, or —OR₃₆, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic. In further embodiments, R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group, a substituted or unsubstituted haloalkyl group, a substituted or unsubstituted hydroxyalkyl group, a substituted or unsubstituted cyanoalkyl group, a substituted or unsubstituted alkenyl group, a substituted or unsubstituted C₁-C₆alkylcarbonyl group, a substituted or unsubstituted alkynyl group, a substituted or unsubstituted cycloalkyl group, a substituted or unsubstituted cycloalkenyl group, a substituted or unsubstituted alkylcycloalkyl group, a substituted or unsubstituted alkylcycloalkenyl group, a substituted or unsubstituted heterocycloalkyl group, a substituted or unsubstituted alkylheterocycloalkyl group, a substituted or unsubstituted heterocycloalkenyl group, a substituted or unsubstituted alkylheterocycloalkenyl group, a substituted or unsubstituted aryl group, or a substituted or unsubstituted heteroaryl group. In more particular embodiments, R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group. In further embodiments, R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, or a substituted or unsubstituted C₁-C₆ alkyl group. In other embodiments, R₁₄, R₁₅, R₁₆R₁₇, R₁₂, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2 hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In other embodiments, R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, t-butyl, 1-pentyl, 2 pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl. In more specific embodiments, R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, or t-butyl.

In more particular embodiments, R₁₃ is selected from hydroxyl group, —NR₁₄R₁₅, or —CR₁₆R₁₇R₁₈, wherein R₁₄, R₁₅, R₁₆R₁₇, and R₁₈ are each independently selected from H, halo group, hydroxyl group, a substituted or unsubstituted thiol group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic. In further embodiments, R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a thiol group, an amino group, a substituted or unsubstituted alkyl group, or —S—C(O)R₃₇, wherein R₃₇ is selected from H or a substituted or unsubstituted alkyl group.

In more particular embodiment, R₁₃ is a hydroxyl group. In another embodiment, R₁₃ is —NR₁₄R₁₅, wherein R₁₄ and R₁₅ are each independently selected from H, halo group, hydroxyl group, a substituted or unsubstituted thiol group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic. In another embodiment, R₁₃ is —NR₁₄R₁₅, wherein R₁₄ and R₁₅ are each independently selected from H, halo group, hydroxyl group, a substituted or unsubstituted thiol group, a substituted or unsubstituted amino group, or a substituted or unsubstituted alkyl group. In another embodiment, R₁₃ is —NR₁₄R₁₅, wherein R₁₄ and R₁₅ are each independently selected from H, hydroxyl group, a thiol group, or a substituted or unsubstituted C₁-C₆ alkyl group. In another embodiment, R₁₃ is —NR₁₄R₁₅, wherein R₁₄ is H or C₁-C₆ alkyl group and R₁₅ is selected from H, hydroxyl group, or a thiol group.

In another embodiment, R₁₃ is —CR₁₆R₁₇R₁₈, wherein R₁₆, R₁₇, and R₁₈ are each independently selected from H, halo group, hydroxyl group, a substituted or unsubstituted thiol group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic. In a further embodiment, R₁₃ is —CR₁₆R₁₇R₁₈, wherein R₁₆, R₁₇, and R₁₈ are each independently selected from H, halo group, hydroxyl group, a thiol group, an amino group, a substituted or unsubstituted alkyl group, or —S—C(O)R₃₇, wherein R₃₇ is selected from H or a substituted or unsubstituted alkyl group. In a further embodiment, R₁₃ is —CR₁₆R₁₇R₁₈, wherein R₁₆ and R₁₇ are each independently selected from H or a substituted or unsubstituted alkyl group and R₁₈ is selected from a halo group, hydroxyl group, a thiol group, an amino group, a substituted or unsubstituted alkyl group, or —S—C(O)R₃₇, wherein R₃₇ is selected from H or a substituted or unsubstituted alkyl group. In another embodiment, R₁₃ is —CR₁₆R₁₇R₁₈, wherein R₁₆ and R₁₇ are each independently selected from H or a substituted or unsubstituted C₁-C₆ alkyl group and R₁₈ is selected from a halo group, hydroxyl group, a thiol group, an amino group, a substituted or unsubstituted C₁-C₆ alkyl group, or —S—C(O)R₃₇, wherein R₃₇ is selected from H or a substituted or unsubstituted C₁-C₆ alkyl group. In another embodiment, R₁₃ is —CR₁₆R₁₇R₁₈, wherein R₁₆ and R₁₇ are each independently selected from H and R₁₈ is selected from a halo group, hydroxyl group, a thiol group, or —S—C(O)R₃₇, wherein R₃₇ is selected from H or a substituted or unsubstituted C₁-C₆ alkyl group.

Certain embodiments of the compounds of Formula I include at least one compound selected from:

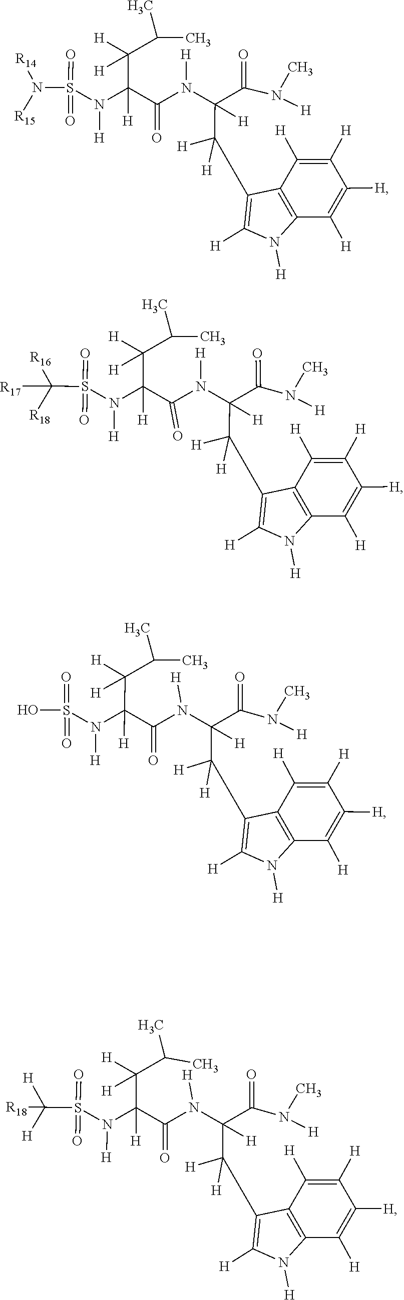

a salt, hydrate, solvate, tautomer, enantiomer, racemate, diastereomer, or combination thereof.

Certain embodiments of the compounds of Formula I include at least one compound selected from:

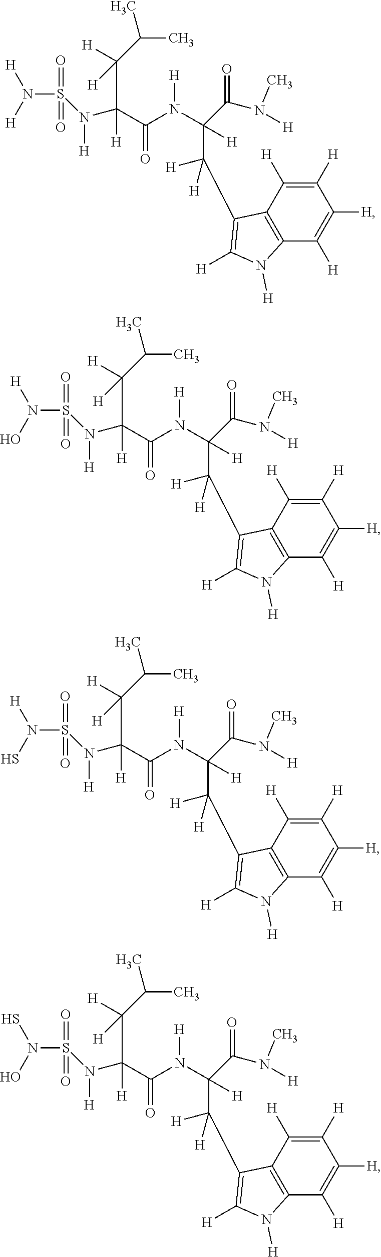

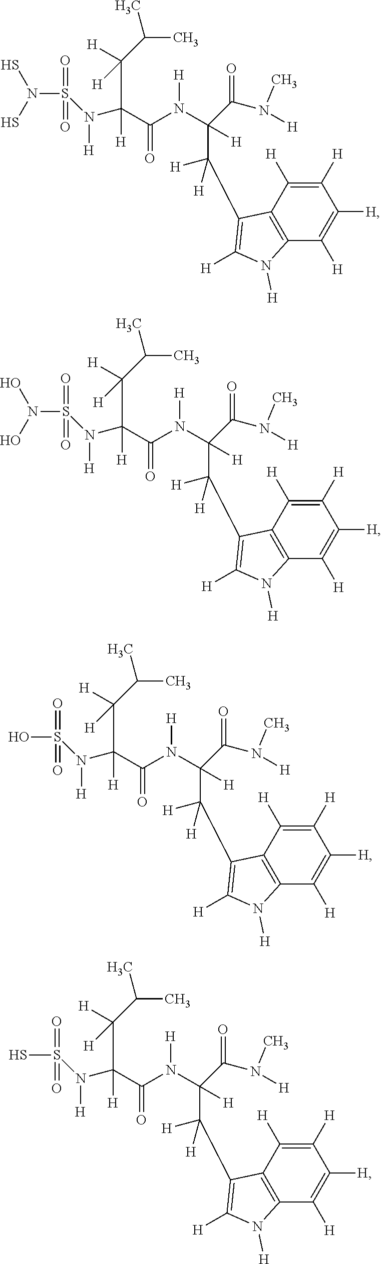

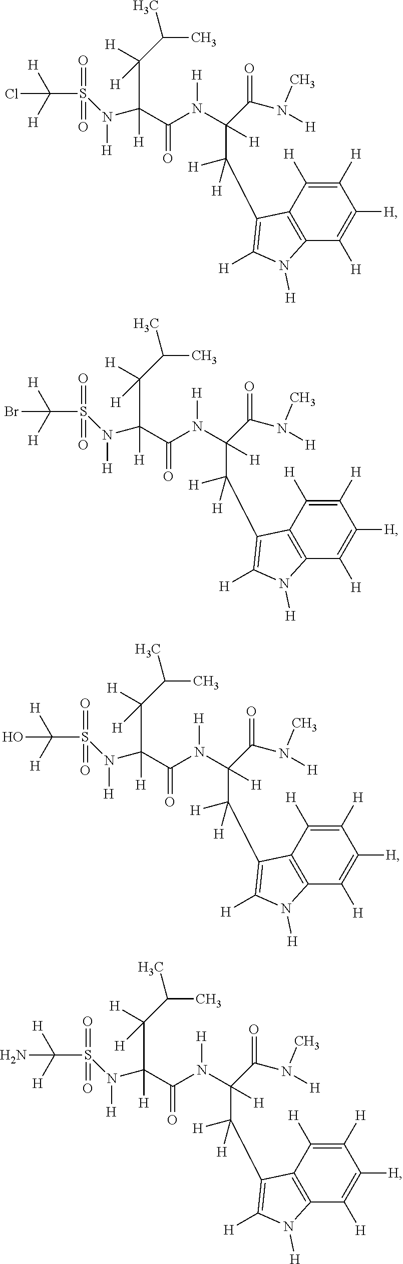

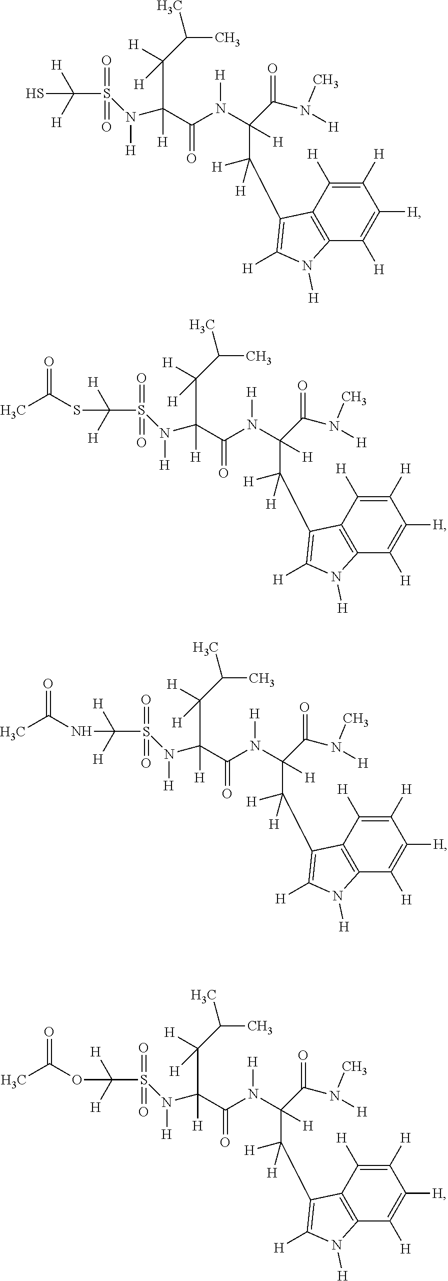

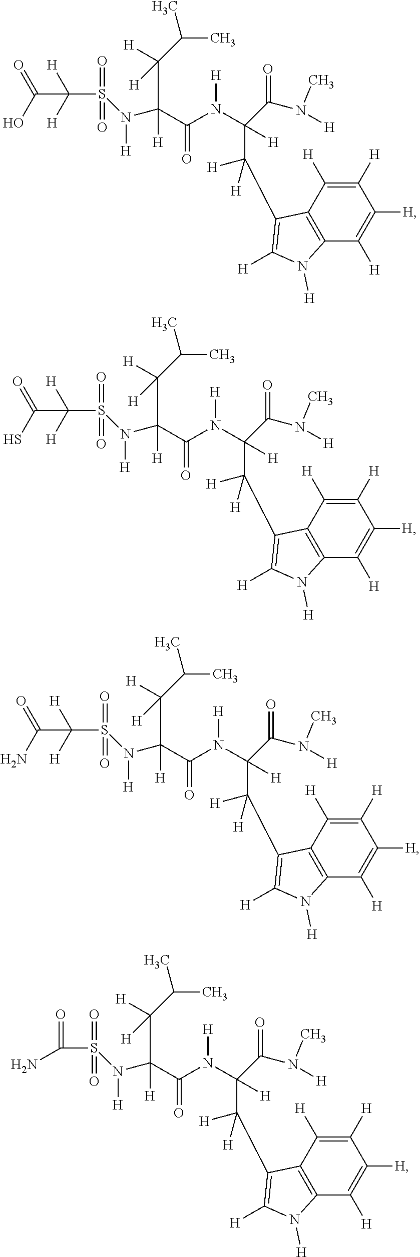

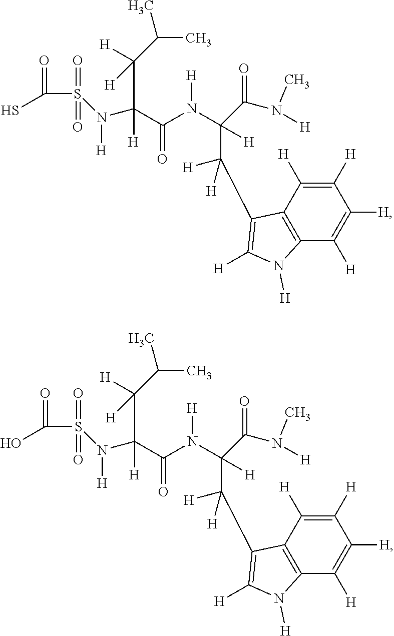

a salt, hydrate, solvate, tautomer, enantiomer, racemate, diastereomer, or combination thereof.

Certain embodiments of the compounds of Formula II include at least one compound selected from:

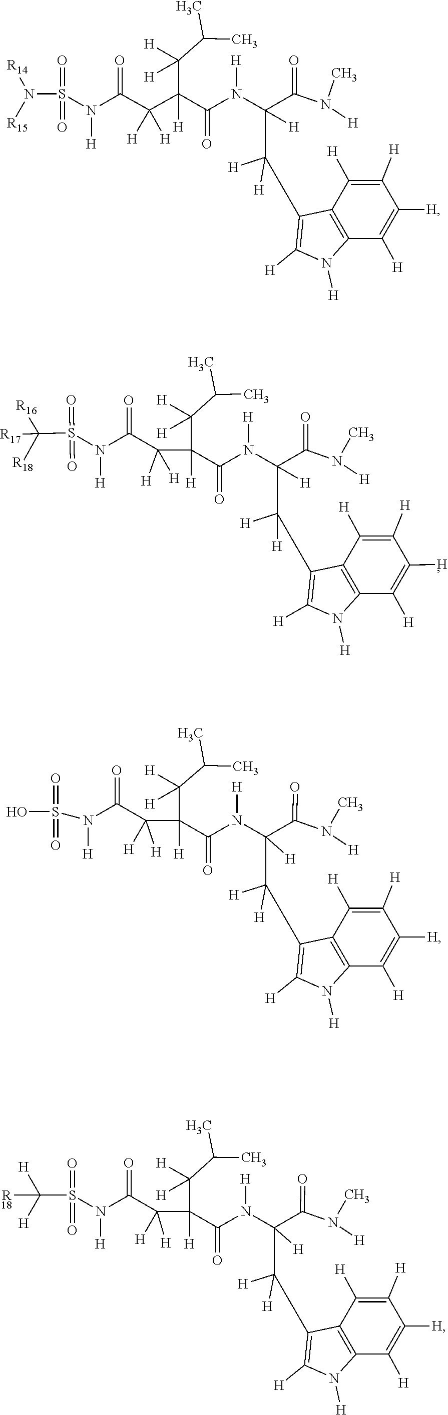

a salt, hydrate, solvate, tautomer, enantiomer, racemate, diastereomer, or combination thereof.

Certain embodiments of the compounds of Formula II include at least one compound selected from:

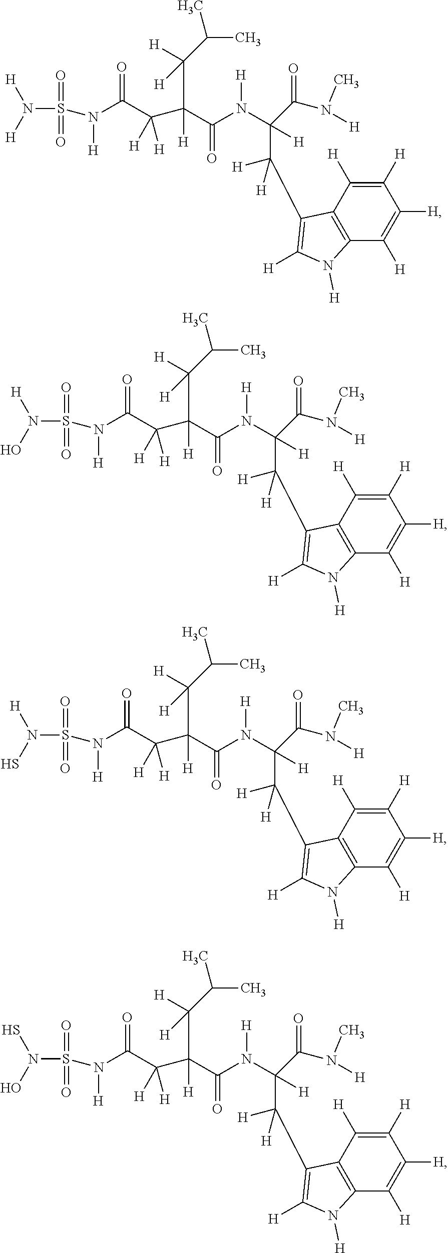

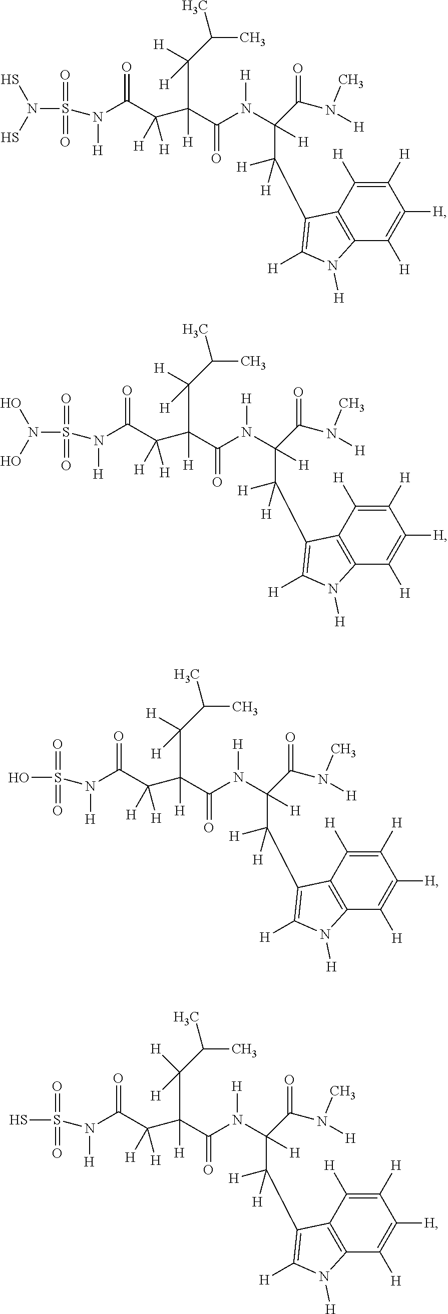

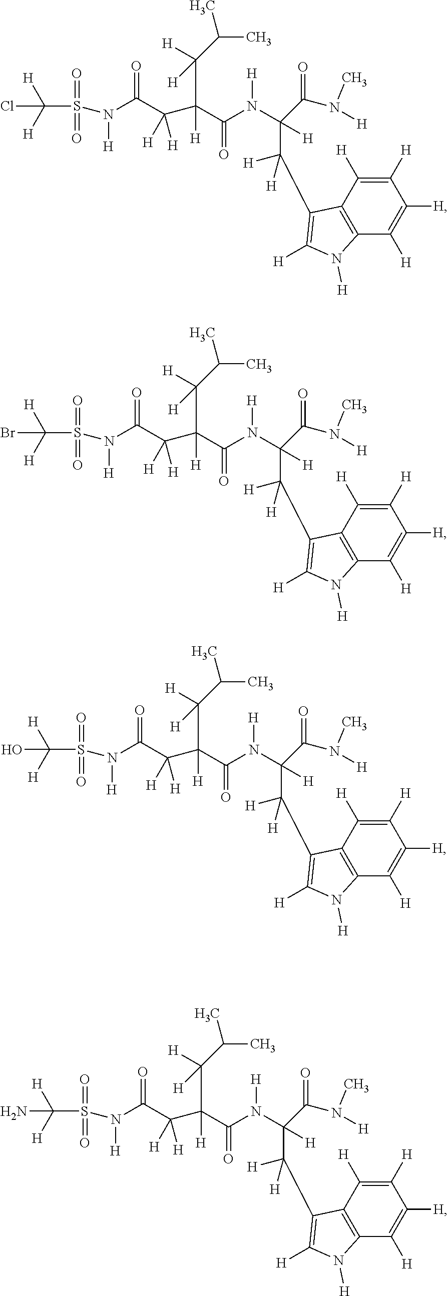

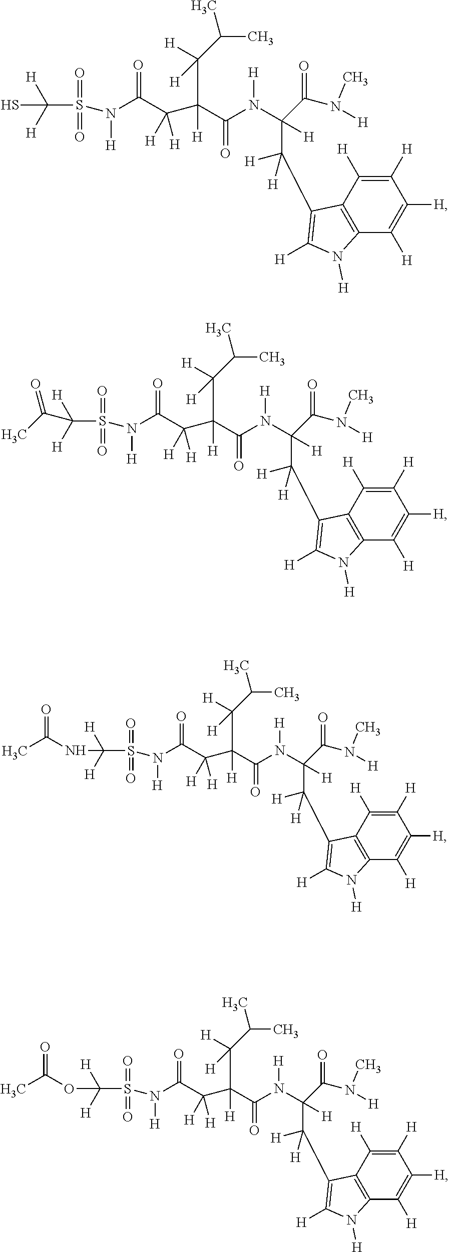

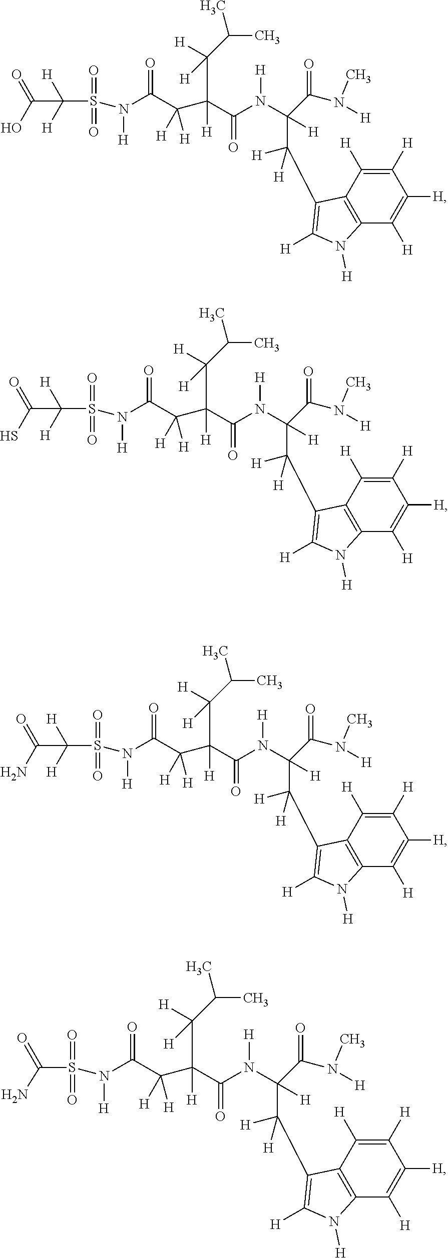

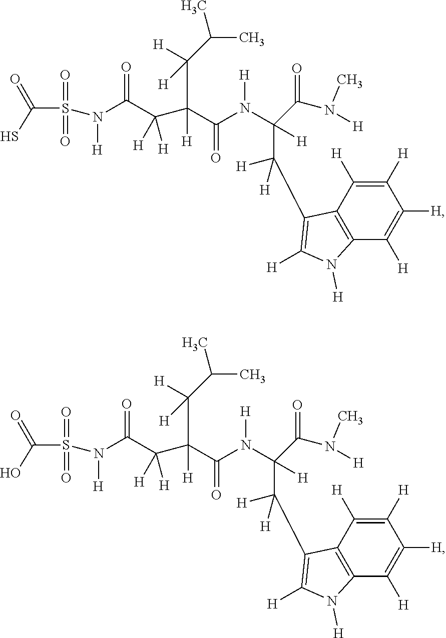

a salt, hydrate, solvate, tautomer, enantiomer, racemate, diastereomer, or combination thereof.

The compounds disclosed herein can be in the form of a pharmaceutically-acceptable salt thereof, a hydrate thereof, a solvate thereof, a tautomer thereof, an enantiomer, racemate, diastereomer thereof, or a combination thereof. In more specific embodiments, the compounds of Formulae I and II can have an S or R configuration at the α-carbon with R₇ and an S or R configuration at the α-carbon with R₁₀ₐ and R_{10b}. In specific embodiments, the compounds of Formulae I and II can have an S configuration at the α-carbon with R₇ and an S configuration at the α-carbon with R₁₀ₐ and R_{10b}. Certain examples of the compounds of Formulae I and II are shown in FIG. 4D as AP-1 to AP-7.

### Method of Making Sulfonamide-Based Inhibitors

The compounds described herein can be made using a variety of methods. In one embodiment of the method, the compound of Formula I can be made as follows and the groups are defined as in the previous section:

a) A compound of Formula IA is reacted with an amine to form an intermediate of Formula IB. R₄₀ can be selected from any suitable group listed in the previous section with respect to the R groups. Typically, R₄₀ is selected from substituted or unsubstituted hydrocarbyl group, for example, a substituted or unsubstituted alkyl group. More particularly, R₄₀ is selected from any suitable substituted or unsubstituted C₁-C₆ alkyl group such as methyl, ethyl, and the like. X⁻ is selected from any suitable counterion, for example, halide ions, NO₃⁻, ClO₄⁻, OH⁻, H₂PO₄⁻, HSO₄⁻, sulfonate ions or carboxylate ions. In another embodiment, wherein X⁻ is selected from Cl⁻, Br or F⁻.

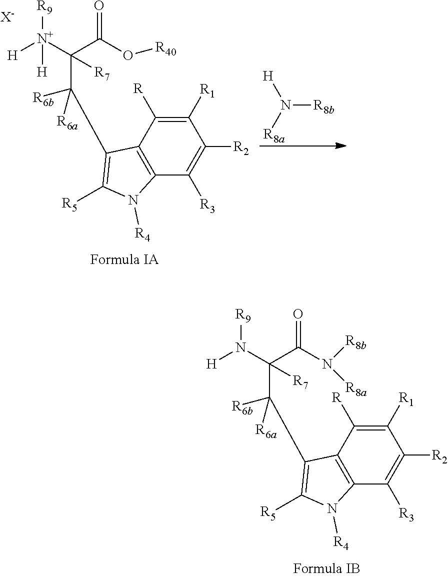

b) The intermediate of Formula IB is reacted with an acid to yield Formula IC. PG is a protecting group. Any suitable protecting group may be used and examples are provided under the definition section.

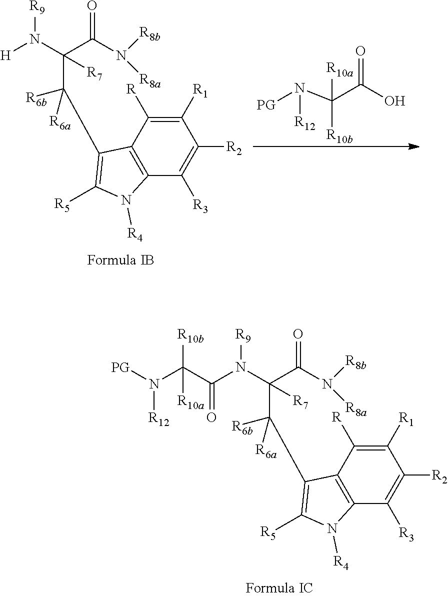

c) The intermediate of Formula IC is deprotected with a suitable base, for example, piperidine, to yield Formula ID.

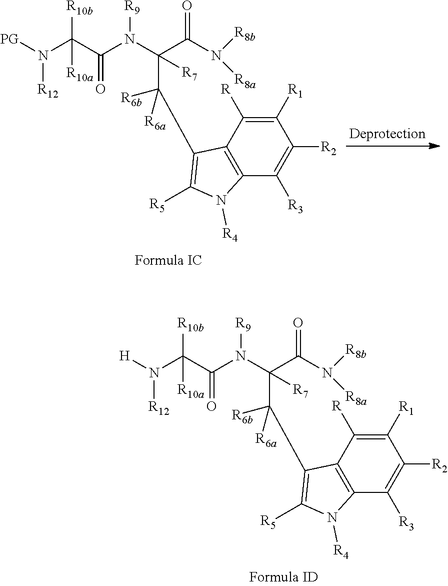

d) The intermediate of Formula ID is reacted with a sulfonyl compound to yield Formula I. LG is any suitable leaving group, for example, a weak base such as halides (e.g., Cl, Br, I), tosylates, mesylates, and perfluoroalkylsulfonates.

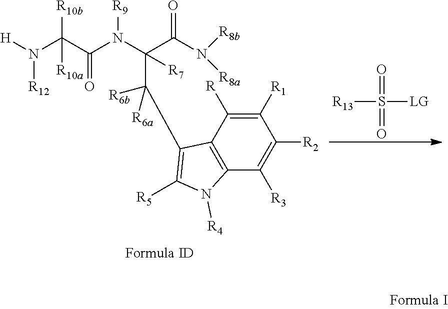

In another embodiment of the method, the compound of Formula II can be made as follows and the groups are defined as in the previous section:

a) A compound of Formula IA is reacted with an amine to form an intermediate of Formula IB. R₄₀ can be selected from any suitable group listed in the previous section with respect to the R groups. Typically, R₄₀ is selected from substituted or unsubstituted hydrocarbyl group, for example, a substituted or unsubstituted alkyl group. More particularly, R₄₀ is selected from any suitable substituted or unsubstituted C₁-C₆ alkyl group such as methyl, ethyl, and the like. X⁻ is selected from any suitable counterion, for example, halide ions, NO₃⁻, ClO₄⁻, OH⁻, H₂PO₄⁻, HSO₄⁻, sulfonate ions or carboxylate ions. In another embodiment, wherein X⁻ is selected from Cl⁻, Br⁻ or F⁻.

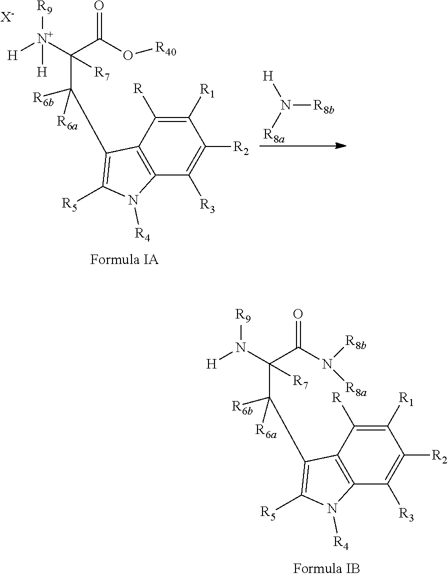

b) The intermediate of Formula IB is reacted with an acid to yield Formula IE. PG is a protecting group. Any suitable protecting group may be used and examples are provided under the definition section.

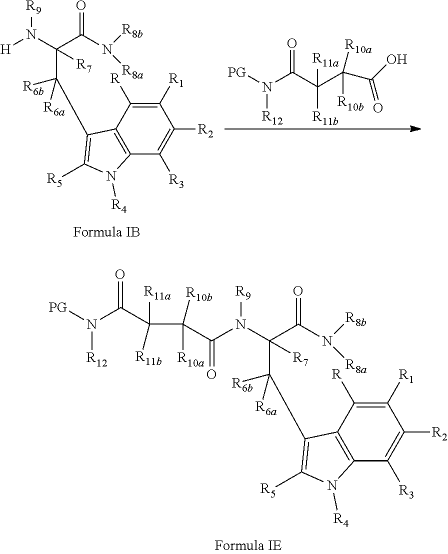

c) The intermediate of Formula IE is deprotected with a suitable base, for example, piperidine, to yield Formula IF.

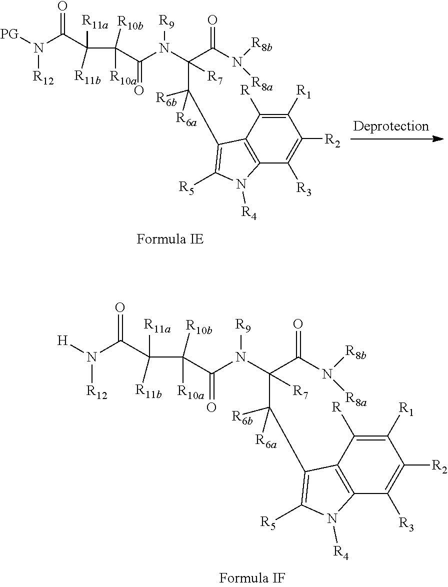

d) The intermediate of Formula IF is reacted with a sulfonyl compound to yield Formula II. LG is any suitable leaving group, for example, a weak base such as halides (e.g., Cl, Br, I), tosylates, mesylates, and perfluoroalkylsulfonates.

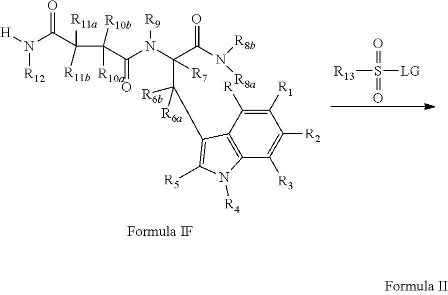

In general, the compounds disclosed herein may be prepared by employing reactions and standard manipulations that are known in the literature or exemplified herein.

### Uses and Methods of Use of Sulfonamide-Based Inhibitors

One or more of the compounds of Formulae I and II disclosed herein, may be used in the treatment of matrix metalloproteinase mediated conditions (e.g. diseases and/or disorders) having excessive ECM degradation and/or remodelling. Examples of the matrix metalloproteinase mediated conditions include cancer, angiogenesis, cardiovascular disease, neurological disease, inflammation, eye disease, autoimmune disease, for regulating contraception, or other conditions that are affected by the regulation of MMPs. In particular, the compounds of Formulae I and II disclosed herein, may be used in the treatment of, for example, MMP-3, MMP-8 and MMP-13 mediated degenerative diseases having excessive ECM degradation and/or remodelling. One or more of the compounds of Formulae I and II disclosed herein, therefore, may exhibit selectivity for one or more specific MMPs.

The cancer can be pancreatic cancer, gastric cancer, lung cancer, colorectal cancer, prostate cancer, renal cell cancer, basal cell cancer, breast cancer, bone cancer, brain cancer, lymphoma, leukemia, melanoma, myeloma and other hematological cancers, and the like. The cancer can be primary, metastatic, or both.

The neurological disease can be one that arises from at least one of painful neuropathy, neuropathic pain, diabetic neuropathy, drug dependence, drug withdrawal, depression, anxiety, movement disorders, tardive dyskinesia, cerebral infections that disrupt the blood-brain barrier, meningitis, stroke, hypoglycemia, cardiac arrest, spinal cord trauma, head trauma, and perinatal hypoxia. The neurological disease can also be a neurodegenerative disorder. The neurological disease can be epilepsy, Alzheimer's disease, Huntington's disease, Parkinson's disease, multiple sclerosis, or amyotrophic lateral sclerosis, as well as Alexander disease, Alper's disease, Ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Kennedy's disease, Krabbe disease, lewy body dementia, Machado-Joseph disease (Spinocerebellar ataxia type 3), Multiple System Atrophy, Pelizaeus-Merzbacher disease, Pick's disease, primary lateral sclerosis, Refsum's disease, S andhoff disease, Schilder's disease, spinocerebellar ataxia (multiple types with varying characteristics), spinal muscular atrophy, Steele-Richardson-Olszewski disease, or tabes dorsalis.

Inflammation diseases can be an inflammation disease that involves connective tissue, airway tissue, or central nervous system tissue. The inflammation can be acute asthma, chronic asthma, allergic asthma, or chronic obstructive pulmonary disease. In one embodiment, the inflammation is arthritis.

Other conditions that may be affected by the regulation of MMPs is skin disease. The compounds disclosed herein can also be used in imaging, wherein the inhibitor can be modified to be detectable by imaging techniques; for pre- and post operative treatments for removal of tumors; and in combination with any other chemotherapeutic modalities (biological and non-biological).

With respect to the treatment of matrix metalloproteinase mediated conditions, the conditions include, for example, cancer (e.g. melanoma, brain tumours (e.g. GBM), gastric carcinoma or non-small cell lung carcinoma), tumor metastasis, angiogenesis in tumors, rheumatoid arthritis, osteoarthritis, abdominal aortic aneurysm, inflammation, atherosclerosis, multiple sclerosis, chronic obstructive pulmonary disease, ocular diseases (e.g. ocular inflammation, glaucoma, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, atherosclerosis, cardiovascular disease, reperfusion injury, trauma, chemical exposure or oxidative damage to tissues, chronic wound healing, wound healing, hemorrhoid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, liver fibrosis, lupus, malaria, meningitis, multi-organ dysfunction, necrotizing enterocolitis, osteoporosis, chronic periodontitis, periodontitis, peritonitis associated with continuous ambulatory peritoneal dialysis (CAPD), pre-term labor, polymyositis, post-surgical trauma, pruritis, psoriasis, psoriatic arthritis, pulmatory fibrosis, pulmatory hypertension, renal reperfusion injury, respiratory viruses (e.g. coronaviruses), restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

With respect to the treatment of matrix metalloproteinase mediated conditions wherein the condition is cancer, infiltration of cancer is reliant on the coordination of the tumour microenvironment. In particular, the ECM is a regulator of cancer cell invasion, migration and proliferation. Identification of genes that are differentially regulated by invasive glioma are of interest. Using a model of glioma migration/invasion based on gap junction protein connexin43 (Cx43) expression (Bates, D. C. et al. Connexin43 enhances glioma invasion by a mechanism involving the carboxy terminus. Glia 2007, 55 (15), 1554-64; Naus, C. C. et al. Common mechanisms linking connexin43 to neural progenitor cell migration and glioma invasion. Semin Cell Dev Bio 12016, 50, 59-66; Kameritsch, P. et al., Channel-independent influence of connexin 43 on cell migration. Biochim Biophys Acta 2012, 1818 (8), 1993-2001; Oliveira, R. et al., Contribution of gap junctional communication between tumor cells and astroglia to the invasion of the brain parenchyma by human glioblastomas. BMC Cell Biol 2005, 6 (1), 7; Sin, W. C. et al., Astrocytes promote glioma invasion via the gap junction protein connexin43. Oncogene 2016, 35 (12), 1504-16; and Aftab, Q. et al., Reduction in gap junction intercellular communication promotes glioma migration. Oncotarget 2015, 6 (13), 11447-64), increases in the proteinase matrix metalloproteinase-3 (MMP3) within the conditioned media (secretome) have been demonstrated (Aftab, Q. et al., Cx43-Associated Secretome and Interactome Reveal Synergistic Mechanisms for Glioma Migration and MMP3 Activation. Front Neurosci 2019, 13, 143). These finding are supported by previous studies demonstrating MMP3 at invasive fronts of GBM tumors, and the reduction of invasion potential with MMP3 loss. (Jin, X. et al., Blockade of EGFR signaling promotes glioma stem-like cell invasiveness by abolishing ID3-mediated inhibition of p27(KIP1) and MMP3 expression. Cancer Lett 2013, 328 (2), 235-42.) Signals mediated by MMPs include the activation/inactivation of growth factors, shedding of cell surface adhesion molecules, and ECM-bound cytokines, growth factors, and cryptic peptides (Lopez-Otin, C. et al., Protease degradomics: a new challenge for proteomics. Nat Rev Mol Cell Biol 2002, 3 (7), 509-19; and Overall, C. M. et al., Strategies for MMP inhibition in cancer: innovations for the post-trial era. Nat Rev Cancer 2002, 2 (9), 657-72). As there is a strong correlation between patients outcome with the activities of proteases within the extracellular space (Fried, P. et al., Tube travel: the role of proteases in individual and collective cancer cell invasion. Cancer Res 2008, 68 (18), 7247-9), in embodiments, cancer (e.g. GBM) processes may be inhibited that are mechanistically dependent upon the MMP.

One or more of the compounds of Formulae I and II for treatment of a condition comprising contacting a cell with a compound of Formulae I and/or II, wherein the compound is effective to inhibit a matrix metalloproteinase. In another embodiment, a compound of Formulae I and/or II for treatment of a subject in need thereof, comprising administering to the subject an effective amount of a matrix metalloproteinase inhibitor of a compound of formulas I-II. The matrix metalloproteinase can be a gelatinase, collagenase, stromelysin, membrane-type MMP, or matrilysin. The matrix metalloproteinase can be, for example, MMP-3, MMP-8, or MMP-13. The matrix metalloproteinase can be a human matrix metalloproteinase.

In embodiments, the compounds of Formulae I and II are useful as active ingredients in pharmaceutical compositions for the treatment or prevention of matrix metalloproteinase mediated conditions (e.g. diseases and/or disorders), and, in particular, the ones disclosed herein, including, for example, MMP-3, MMP-8 and MMP-13. One or more of the compounds disclosed herein may be used in pharmaceutical compositions for oral or parenteral administration, including the intravenous, intramuscular, intraperitoneal, and subcutaneous routes of administration.

Methods of inhibiting matrix metalloproteinases by administering formulations/compositions comprising one or more of the compounds disclosed herein for the treatment of conditions (e.g. diseases) or symptoms arising from or associated with matrix metalloproteinase (e.g. MMP-3, MMP-8 and MMP-13), including prophylactic and therapeutic treatment. The formulations may include, for example, oral, rectal, topical, intravenous, parenteral (e.g. intramuscular, intravenous), ocular (e.g. ophthalmic), transdermal, inhalative (e.g. pulmonary, aerosol inhalation), nasal, sublingual, subcutaneous or intraarticular formulations. The suitable route in any given case will depend on the nature and severity of the conditions being treated and on the nature of the active ingredient. The compounds of Formulae I and II can be used in suitable unit dosage forms and prepared according to standard pharmaceutical practice. The formulations/compositions may include an effective amount of one or more of the compounds of Formulae I and II and a pharmaceutically acceptable carrier. The compounds of Formulae I and II may be administered to mammals, typically humans, either alone or, in combination with pharmaceutically acceptable carriers or diluents, optionally with known adjuvants, such as alum, in a pharmaceutical composition, according to standard pharmaceutical practice.

One or more of the compounds of Formulae I and II may be advantageously administered orally, unlike most current cancer therapies, which are administered intravenously. For oral use of a compound or composition, the selected compound may be administered, for example, in the form of tablets or capsules, or as an aqueous solution or suspension. In the case of tablets for oral use, carriers which are commonly used include lactose and corn starch, and lubricating agents, such as magnesium stearate, are commonly added. For oral administration in capsule form, useful diluents include lactose and dried corn starch. When aqueous suspensions are required for oral use, the active ingredient is combined with emulsifying and suspending agents. If desired, certain sweetening and/or flavoring agents may be added. For intramuscular, intraperitoneal, subcutaneous and intravenous use, sterile solutions of the active ingredient are usually prepared, and the pH of the solutions should be suitably adjusted and buffered. For intravenous use, the total concentration of solutes should be controlled in order to render the preparation isotonic.

When one or more of the compounds of Formulae I and II is administered into a human subject, the daily dosage will normally be determined by the prescribing physician with the dosage generally varying according to the age, weight, and response of the individual patient, as well as the severity of the patients symptoms. In one exemplary application, a suitable amount of compound is administered to a mammal undergoing treatment for cancer. Administration occurs in an amount from about 0.01 mg/kg of body weight to greater than about 100 mg/kg of body weight per day; from about 0.01 mg/kg of body weight to about 500 mg/kg of body weight per day; from about 0.01 mg/kg of body weight to about 250 mg/kg of body weight per day; or 0.01 mg/kg of body weight to about 100 mg/kg of body weight per day. These dosages can be more particularly used orally.

In embodiments, the compounds of Formulae I and II may be used to inhibit MMP-3 and methods of treating conditions or symptoms mediated by an MMP-3 enzyme. Such methods include administering one or more compounds of Formulae I and II. Examples of diseases or symptoms mediated by an MMP-3 enzyme include, but are not limited to, cancer, rheumatoid arthritis, osteoarthritis, abdominal aortic aneurysm, inflammation, atherosclerosis, multiple sclerosis, chronic obstructive pulmonary disease, ocular diseases, neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and f[iota]brotic syndromes, intestinal bowel syndrome, allergies, Alzheimers disease, arterial plaque formation, viral infection, stroke, atherosclerosis, cardiovascular disease, reperfusion injury, trauma, chemical exposure or oxidative damage to tissues, pain, inflammatory pain, bone pain and joint pain.

In embodiments, one or more of the compounds of Formulae I and II are useful in the treatment of cancer. The cancer treated may be, for example, brain cancer (e.g. GBM), lung cancer (e.g. small cell or non-small cell lung cancer), cervical cancer, ovarian cancer, cancer of CNS, skin cancer, prostate cancer (e.g. hormone resistant prostate cancer), sarcoma, breast cancer, leukemia, colorectal cancer, neck cancer, lymphoma, pancreatic cancer, gastric cancer, or kidney cancer. More typically, the cancer may be brain cancer, small cell lung cancer, breast cancer (e.g. hormone resistant breast cancer), acute leukemia, chronic leukemia, colorectal cancer. The cancer may be a carcinoma. The carcinoma may be selected from small cell carcinomas, cervical carcinomas, glioma, astrocytoma, prostate carcinomas, ovarian carcinomas, melanoma, breast carcinomas, or colorectal carcinomas.

Compounds of the present invention can have an IC₅₀ for a cancer cell population of less than or equal to about 10,000 nM. In specific embodiments, compounds of the present invention show efficacy against C6 glioma cells at IC₅₀'s of less than about 1000 μM, typically less than about 800 μM, more typically less than about 500 μM.

One or more of the compounds of Formulae I and II disclosed herein and the formulations/compositions thereof, may also include other therapeutic agents that are compatible with one or more of the compounds of Formulae I and II disclosed herein. The therapeutic agents can include, for example, an anti-cancer agent.

Examples of anti-cancer agents include, without being limited thereto, the following: estrogen receptor modulators, androgen receptor modulators, retinoid receptor modulators, cytotoxic agents, antiproliferative agents, tyrosine kinase inhibitors, prenyl-protein transferase inhibitors, HMG-CoA reductase inhibitors, HIV protease inhibitors, reverse transcriptase inhibitors, other angiogenesis inhibitors and combinations thereof. The compounds of Formulae I and II may also be useful with other therapies such as when co-administered with radiation therapy.

“Estrogen receptor modulators” refers to compounds which interfere or inhibit the binding of estrogen to the receptor, regardless of mechanism. Examples of estrogen receptor modulators include, but are not limited thereto, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylomithine, ILX23-7553, trans-N-(4′-hydroxyphenyl) retinamide and N-4-carboxyphenyl retinamide.

“Cytotoxic agents” refer to compounds which cause cell death primarily by interfering directly with the cell's functioning or inhibit or interfere with cell myosis, including alkylating agents, tumor necrosis factors, intercalators, microtubulin inhibitors, and topoisomerase inhibitors. Examples of cytotoxic agents include, but are not limited thereto, cyclophosphamide ifosfamide, hexamethylmelamine, tirapazimine, sertenef, cachectin, ifosfamide, tasonermin, lonidamine, carboplatin, mitomycin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine) platinum, benzylguanine, glufosfamide, GPX100, (trans, trans, trans)-bis-mu-(hexane-1,6-diamine)-mu-[diamine-platinum(II)]bis[diamine(chloro)-platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, and 4-demethoxy-3-deamino-3-aziridinyl-4-methylsulphonyl-daunor-ubicin (see International Patent Application No. WO 00/50032). Examples of microtubulin inhibitors include paclitaxel (Taxol®), vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N—(-3-fluoro-4-methoxyphenyl) benzene sulfonamide, anhydrovinblastine, N,N-dimethyl-L-valyl-L-valyl-N-methyl-L-valyl-L-prolyl-L-proline-t-butylamide, TDX258, and BMS 188797.

Some examples of topoisomerase inhibitors are topotecan, hycaptamine, irinotecan, rubitecan, 6-ethoxypropionyl-3′,4′-O-exo-benzylidene-chartreusin, 9 methoxy-N,N-dimethyl-5-nitropyrazolo[3,4,5-kl]acridine-2-(6H)propanamine, 1-amino-9-ethyl-5-fluoro-2,3-dihydro-9-hydroxy-4-methy-1H, 12H benzo[de]pyrano[3′,4′:b,7]indolizino[1,2b]quinoline-10,13(9H,15H) dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazo-le-1-carboxamide, asulacrine, (5a, 5aB, 8aa, 9b)-9-[2-[N-[2-(dimethylamino)-ethyl]-N-methylamino]ethyl]-5-[4-Hydroxy-3,5-dimethoxyphenyl]-5,5a,6,8,8a,-9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridiniu-m, 6,9 bis[(2-aminoethyl)amino]benzo[g]isoguinoline-5,10-dione, 5-(3-aminopropylamino)-7,10-dihydroxy-2-(2-hydroxyethylaminomethyl)-6H-py-razolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acrid-ine-4-carboxamide, 6-[[2-(dimethylamino)ethyl]amino]-3-hydroxy-7H-indeno[2-, 1-c]quinolin-7-one, and dimesna.

“Antiproliferative agents” includes BCNU, antisense RNA and DNA oligonucleotides such as G3139, ODN698, RVASKRAS, GEM231, and INX3001, and antimetabolites such as floxuridine, enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′ fluoromethylene-2′-deoxy-cytidine, N-[5-(2,3-dihydro-benzofuryl)sulfonyl]-N′-(3,4-dichlorophenyl) urea, N6-[4-deoxy-4-[N2-[2(E),4(E)-tetradecadienoyl]glycylamino]-L-glycer-o-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, and 3-aminopyridine-2-carboxaldehyde thiosemicarbazone.

“Antiproliferative agents” also includes monoclonal antibodies to growth factors, other than those listed under “angiogenesis inhibitors”, such as trastuzumab, and tumor suppressor genes, such as p53, which can be delivered via recombinant virus-mediated gene transfer (see U.S. Pat. No. 6,069,134, for example).

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino-)-7-methoxy-6-[3-(4-morpholinyl)propoxyl]-quinazoline, N-(3-ethynylphenyl)-6,7-bis(2-methoxyethoxy)-4-quinazolinamine, 2,3,9,10,11,12-hexahydro-10-(hydroxymethyl)-10-hydroxy-9-methyl-9,12-epoxy-1H-diindolo[1,2,3-fg: 3′,2′,1′-kl]pyrrolo[3,4-i][1,6]benzodiazocin-1-one, SH1382, genistein, 4-(3 chlorophenylamino)-5,6-dimethyl-7H-pyrrolo[2,3-d]pyrimidinemethane sulfonate, 4-(3-bromo-4-hydroxyphenyl)-amino-6,7-dimethoxyquinazoline, 4-(4′-hydroxyphenyl)amino-6,7-dimethoxyquinazoline, N-4-chlorophenyl-4-(4-pyridylmethyl)-1-phthalazinamine, and Tarceva® (erlotinib).

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Compound AP-1 Synthesis:

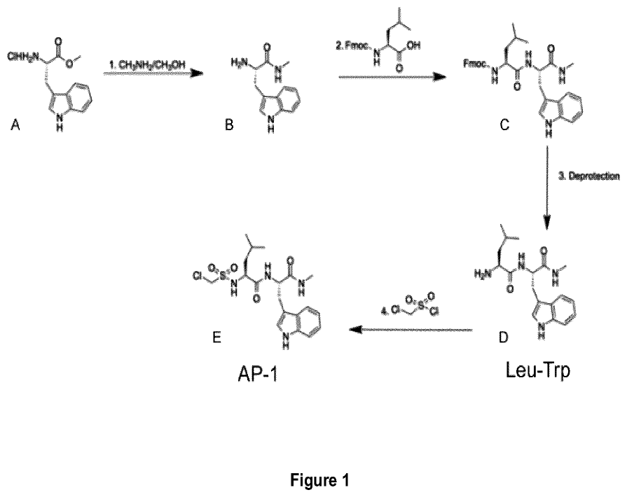

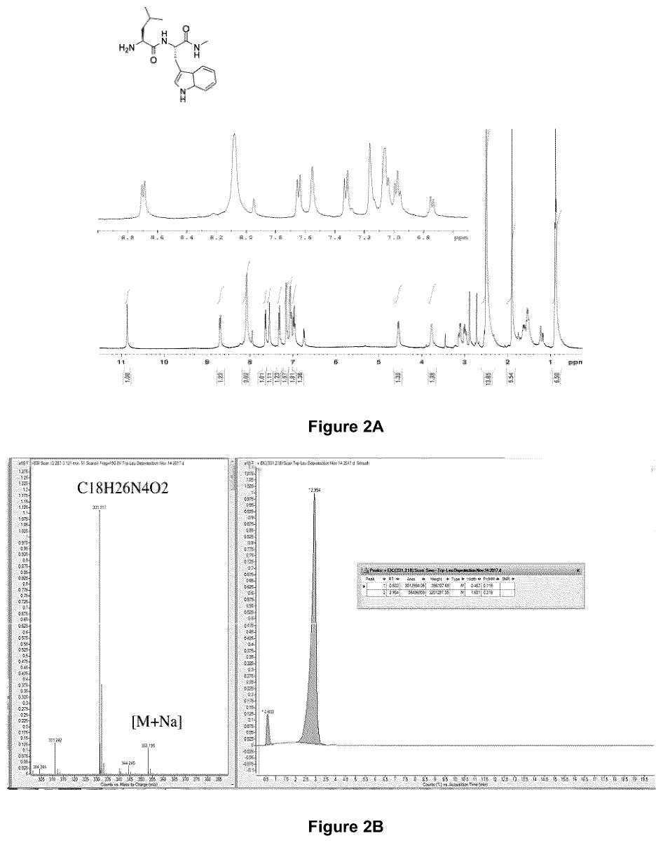

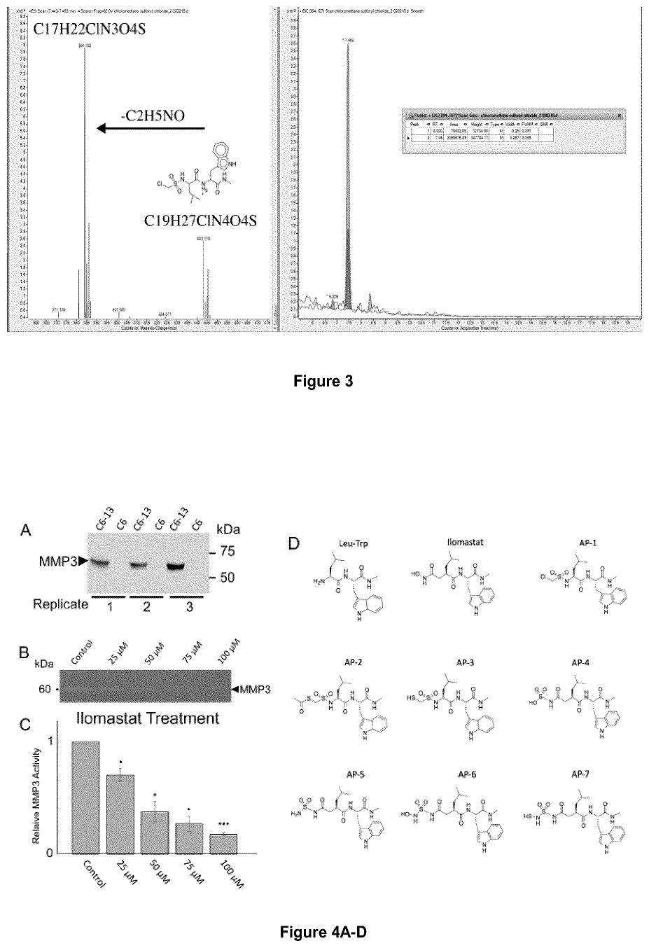

The synthetic scheme is shown in FIG. 1. All starting materials and reagents were commercially available from Sigma-Aldrich and used without further purification. Reactions were monitored via TLC with 210-270 μm silica gel plates (EMD Chemicals Inc., 5715-7) using UV light and potassium permanganate. Flash column chromatography was performed using 230-400 mesh ultrapure silica gel (60 Å, Silicycle). Proton NMR of Compound D was performed on a Bruker spectrometer (400 MHz) with DMSO-d6 as the solvent (FIG. 2A-2B). HPLC-MS was performed, analysis of the Compound AP-1 by QTOF mass spectrometry (Agilent, 6530) (FIG. 3).

Synthesis of Compound B: 0.5062 g (1.963 mmol) L-tryptophan methyl ester hydrochloride (A) in 2.5 mL of McNH₂/MeOH (33% MeNH₂ by wt.) was mixed overnight under nitrogen. Product was then place on rotovap to evaporate MeOH and MeNH₂ to yield L-tryptophan methyl amide (B) as a yellow oil (0.4126 g, 95.6%).

Synthesis of Compound C: 1.387 g (3.926 mmol) Fmoc-Leucine-OH was dissolved in 8 mL of 1:1 DMF:CH₂Cl₂ and mixed with 0.61 mL of DIC, 0.557 g of Oxyma Pure, and 0.70 mL of N,N-Diisopropylethylamine under nitrogen at room temperature for 10 minutes. The solution was then added to 0.4126 g (1.899 mmol) Compound B and mixed for 48 hours under nitrogen. Compound C was purified by flash column chromatography using 2:1 chloroform to methanol as the mobile phase. Fractions were collected, and solvent evaporated to yield a yellow-orange oil (0.8126 g, 77.4%).

Synthesis of Compound D (Leu-Trp): 0.08126 g (0.147 mmol) FMOC-Leucine-Tryptophan was mixed with 4 mL of 20% piperidine in DMF for two hours. Compound D was purified by flash column chromatography using 2:1 chloroform to methanol as the mobile phase. Fractions were collected, and solvent evaporated to yield a yellow-orange oil (0.041 g, 83.9%, C₁₋₈H₂₆N₄O₂, EM: 330.2055, LC-MS m/z: 331.217 (M+H), 353.195 (M+Na), NMR 400 MHz: ˜11 ppm H on N of Trp five carbon ring, ˜7 ppm Trp aromatic H, ˜1 ppm H on methyl groups of Leu).

Synthesis of Compound E (AP-1): 0.78 mL (8.627 mmol) of chloromethane sulfonyl chloride was added to 0.4097 g (1.23 mmol) of Compound D. 1.55 mL (8.627 mmol) of N,N-Diisopropylethylamine was then added and the mixture was refluxed under nitrogen overnight. Compound E was purified by flash column chromatography using 2:1 chloroform to methanol as the mobile phase. Fractions were collected, and solvent evaporated to yield a reddish-orange oil (0.5154 g, 94.5%, C₁₉H₂₇ClN₄O₄S, EM: 442.1442, LC-MS m/z: 443.175 (M+H), 384.103 (M+H, metastable/in-source fragmentation, —C₂H₅N0).

Synthesis of Compound F (AP-2): 0.09855 g (0.8629 mmol) of potassium thioacetate was mixed with 8 mL of THE until dissolved. The solution was then added to 0.0764 g (0.1726 mmol) of Compound E (AP-1) and refluxed under nitrogen for 48 hours. Compound F (AP-2) was purified by flash chromatography using 2:1 chloroform to methanol as the mobile phase. Fractions were collected, and the solvent evaporated to yield a reddish-brown oil (0.0443 g, 54.9%, C₂₁H₃₀N₄O₅S₂, EM: 482.1658, LC-MS m/z: 467.1723 (M-CH₃))

Computational Methods. The 2016 version of MOE (Molecular Operating Environment) was used for all calculations and analysis discussed herein (Molecular Operating Environment (MOE), C.C.G.I., 1010 Sherbrooke St. West, Suite #910, Montreal, QC, Canada, H3A 2R7, 2016.) As a model the crystal structure of MMP-3 with co-crystalized NNGH (PDB: 4G9L) was used. Using the Preparation Tool in MOE the alpha chain was deleted, hydrogens were added and protonation states of the ionizable amino acids were corrected. The protein was then solvated using a periodic boundary condition with NaCl counter ions added at a 0.1 mol/L concentration. The system was then energy minimized to a gradient of 0.1 kcal/mol Å² where the Amber10: EHT force field was used. The minimized system was then ran for a 1.0 ns MD simulation using the NPA algorithm to allow the system to relax. A time step of 2 fs was used. Light bonds were constrained and rigid waters were used. Following the MD simulation the final structure where NNGH was deleted was used for all subsequent calculations.
For the docking a two-step approach was used; the first step involved the use of the triangle matcher placement method to generate random poses. Each pose was then scored using the London dG scoring function where the top 30 scoring poses were kept. In the second step, the 30 poses were then refined using the induced fit method in MOE and rescored using the Generalized-Born Volume Integral/Weighted Surface Area (GBVI/WSA) dG scoring function where the top five scoring conformers were kept.

The compounds docked are listed in Table 1.

TABLE 1
The binding affinities and Zn coordination for ilomastat and various
derivatives. R represents the Trp-Leu backbone of ilomastat.

| Compound | Zinc Binding Group (ZBG)^{a} | Score (kcal mol⁻¹) |
|---|---|---|
| Ilomastat | 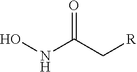 | −12.1 |
| Leu-Trp | 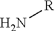 | −8.3 |
| AP-1 | 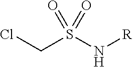 | −7.7 |
| AP-2 | 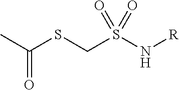 | −8.6 |
| AP-3 | 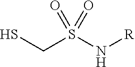 | −11.2 |
| AP-4 | 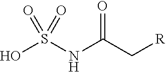 | −9.6 |
| AP-5 | 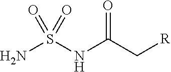 | −9.3 |
| AP-6 | 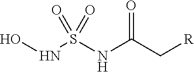 | −12.3 |
| AP-7 | 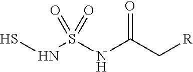 | −11.6 |

^{a}R is the Leu-Trp backbone. See Figure 1 (Compound D) for the structure.

For the docking calculations the receptor was defined to be MMP-3 include the Zn²⁺ ion. As discussed in Jacobsen, J. A.; Major Jourden, J. L.; Miller, M. T.; Cohen, S. M., To bind zinc or not to bind zinc: an examination of innovative approaches to improved metalloproteinase inhibition. Biochim Biophys Acta 2010, 1803 (1), 72-94, favoured ZBGs are hydroxamic acids due to the formation of trigonal bipyrimidal coordination geometries around the Zn²⁺ ion. Moreover, the chelation of the hydroxamic acids to the Zn²⁺ ion enhances the acidity of the ZBG resulting in deprotonation of the OH group enhancing the binding of the ligands to the Zn²⁺ center. The deprotonated OH group is further stabilized by forming a hydrogen bond interaction to the backbone neutral carboxylic side chain of Glu202 (numbering taken from PDB: 4G9L) (Jacobsen, J. A.; Major Jourden, J. L.; Miller, M. T.; Cohen, S. M., To bind zinc or not to bind zinc: an examination of innovative approaches to improved metalloproteinase inhibition. Biochim Biophys Acta 2010, 1803 (1), 72-94; and Belviso, B. D.; Caliandro, R.; Siliqi, D.; Calderone, V.; Arnesano, F.; Natile, G., Structure of matrix metalloproteinase-3 with a platinum-based inhibitor. Chemical communications (Cambridge, England) 2013, 49 (48), 5492-4). Thus, for ilomastat, AP-3, AP-4, AP-6, and AP-7 the acidic protons were removed prior to docking. For Leu-Trp, AP-1, AP-2, and AP-5 no acidic protons are present. For all compounds Glu202 was modelled as neutral.

For the ligands that had predicted Gibbs binding energies comparable to those for ilomastat, MD simulations were run to investigate the effect of dynamics on the Gibbs binding energies. For these MD simulations to reduce computational costs, the atoms in the first two environmental shells surrounding the ligand were free to move whereas the atoms in the third environmental shell and beyond were tethered. The whole protein-ligand complex was then solvated using a droplet boundary condition with a margin of 10. The waters were held in place by adding a potential wall with a weight of 100 to maintain the shape of the droplet. The system was minimized using the AMBER10: EHT FF to gradient of 0.1 kcal/mol Å². Following the minimization, the systems were then ran for a 10 ns MD production simulation using the NPA algorithm. A time step of 2 fs was used. Light bonds were constrained and rigid waters were used.

Glioma Cell Culture. The C6 glioma line (American Type Culture Collection, ATCC) maintains many characteristics of GBM including cancer stem cell behavior ability to generate GBMs in rats upon injection in the brain (Grobben, B.; De Deyn, P. P.; Slegers, H., Rat C6 glioma as experimental model system for the study of glioblastoma growth and invasion. Cell Tissue Res 2002, 310 (3), 257-70; Zheng, X.; Shen, G.; Yang, X.; Liu, W., Most C6 cells are cancer stem cells: evidence from clonal and population analyses. Cancer Res 2007, 67 (8), 3691-7; and Beljebbar, A.; Dukic, S.; Amharref, N.; Manfait, M., Ex vivo and in vivo diagnosis of C6 glioblastoma development by Raman spectroscopy coupled to a microprobe. Anal Bioanal Chem 2010, 398 (1), 477-87). The C6 clone stably expressing Cx43 demonstrates enhanced gap junctional intercellular communication, and restricted proliferation, with enhanced migratory potential (scrape-wound assay) when compared to wild-type C6 (Aftab, Q.; Mesnil, M.; Ojefua, E.; Poole, A.; Noordenbos, J.; Strale, P. O.; Sitko, C.; Le, C.; Stoynov, N.; Foster, L. J.; Sin, W. C.; Naus, C. C.; Chen, V. C., Cx43-Associated Secretome and Interactome Reveal Synergistic Mechanisms for Glioma Migration and MMP3 Activation. Front Neurosci 2019, 13, 143; and Naus, C. C.; Zhu, D.; Todd, S. D.; Kidder, G. M., Characteristics of C6 glioma cells overexpressing a gap junction protein. Cell Mol Neurobiol 1992, 12 (2), 163-75.) Glioma cells were maintained at 37° C., 95% air and 5% CO₂ in a humidified incubator. Cells were cultivated in high glucose DMEM (4.0 g/L, ThermoFisher Scientific) containing L-glutamine (4.0 mM), antibiotics (penicillin, 100 IU/mL; streptomycin, 100 μg/mL) and 10% fetal bovine serum (FBS; Gibco). For collection of conditioned media, cells were plated in 6-well plate (200,000 cells/well) and grown to 80% confluency over the course of 3 days. At this time 3 mL of serum-free DMEM was added to each well and incubated for 24 hours. For zymography, serum free medium was prepared with ilomastat (Selleck Chemical, S7157, purity >99.15%, 0, 25, 50, 75, or 100 μM). For NFF-3 experiments cells were grown to 80 90% confluence in 145 mm diameter plates, with DMEM was used for conditioning.
Zymography. The conditioned media was concentrated using spin filters, with the tubes being centrifuged at 4800 g for 12 minutes in pre-chilled rotors. Protein concentration was determined by BCA assay and samples suspended in SDS loading buffer (no DTT and without boiling). Protein 100 μg/well were separated in 10% SDS-PAGE gels supplemented with 0.1% gelatin (125V, ˜3 hours). The gel was then washed twice in 45 minutes each in incubation buffer (50 mM Tris-HCl, pH=7.5, 5 mM CaCl₂, 1 uM ZnCl₂, 0.02% Brij-35, 0.02% NaN₃, and 2.5% Triton X-100). The gel was washed in incubation buffer (50 mM Tris-HCl, pH=7.5, 5 mM CaCl₂, 1 uM ZnCl₂, 0.02% Brij-35 and 0.02% NaN₃) which was replaced after 10 minutes. In a sealed container, gels were incubated at 37° C. for 20 hours, followed by staining (Coomassie). Gels were processed in water to reveal the presence of activated MMPs as light bands against dark blue gel.
SDS-PAGE/Western Blotting. Conditioned media was collected and chilled on ice. Proteins were precipitated using cold acetone and chilled (−20° C., 1 hour). Proteins were pelleted by centrifugation (12 minutes, 4800 g). Proteins were suspended in SDS-PAGE loading buffer. A small amount of this material was diluted and taken for quantification by BCA. Protein samples were loaded on a 10% SDS-PAGE gel and separated under constant voltage (125V). Proteins were then transferred at 30V for 3 hours at 4° C., and blocked using 5% milk (TBST, 1 hour). Membranes were incubated at 4° C. primary antibody (1:1000 rabbit antiMMP-3, ProteinTech, 1% milk in TBST) overnight. Membranes were washed 3× 15 min with TBST, followed HRPO-conjugated secondary antibody (1:2000, 1% milk, 1 hour, room temperature). The membrane was washed three times with TBST (3×15 min) prior to ECL imaging (Li-Cor, CDigit) and quantification (ImageJ).
NFF-3 Fluorescence Assay. Conditioned media was then collected and distributed into a 96-well assay plate preloaded with 15 μL of a stock solution containing NFF-3 (100 μM in DMSO). Conditioned or control media (300 μL) was added to each well (final concentration NFF-3=4.76 μM, MW=1675.8 g/mole) using a standard 96 well plate. MMP3 activity was monitored at 37° C. using a multi-well plate fluorimeter (SpectraMax M2, Molecular Devices). The proteolytic activity of C6-13 conditioned media was measured relative to the background fluorescence produced by unconditioned DMEM. Wavelengths for excitation (325 nm) and fluorescence emission (393 nm) were measured every 10 min over 6 h. The NFF-3 probe is from Cayman Chemical (395% pure, MW: 1675.8). After conditioning, the media was collected. Each experimental condition had its own control condition. Using a 96 well plate 15 μL of distilled water were added to the control wells and 15 μL of 100 μM NFF-3 were added to the experimental well. Then 300 μL of the conditioned media (C613, and DMEM media) were added to the control and experimental wells, resulting in an overall concentration of 4.76 μM NFF-3. The excitation wavelength of the spectrometer was set to 325 nm while the emission was set to 393 nm. The fluorescence was measured every ten minutes over the course of six hours, with the plate shaking for 15 seconds before each reading. The experiment was conducted at 37° C.
Inhibition of MMP3 in the Conditioned Media of C6-13 Glioma. Connexin43 (Cx43) has been shown to promote migration by wound healing, Transwell™ assay, and brain slices (Bates, D. C. et al., Connexin43 enhances glioma invasion by a mechanism involving the carboxy terminus. Glia 2007, 55 (15), 1554-64 and Oliveira, R. et al., Contribution of gap junctional communication between tumor cells and astroglia to the invasion of the brain parenchyma by human glioblastomas. BMC Cell Biol 2005, 6 (1), 7). Increased motility due to Cx43 expression was found to have correlated increases in MMP3 (Aftab, Q. et al., Cx43-Associated Secretome and Interactome Reveal Synergistic Mechanisms for Glioma Migration and MMP3 Activation. Front Neurosci 2019, 13, 143). Demonstrating a direct link to secreted factors, the transfer of this material was sufficient to stimulate movement in slower moving cells (Aftab, Q. et al., Cx43-Associated Secretome and Interactome Reveal Synergistic Mechanisms for Glioma Migration and MMP3 Activation. Front Neurosci 2019, 13, 143). SDS-PAGE/westernblot analysis of MMP3 was performed within the conditioned media from C6-Cx43, and C6 (parental line) serving as negative controls (FIG. 4A). In biological triplicates, analysis of conditioned media observed binary increases of MMP3 within the conditioned media of C6-Cx43 cells. Zymographic assays incorporating natural substrate (0.1% gelatin) demonstrated the concentration-dependent inhibition of secreted MMP3 in glioma exposed to varying ilomastat concentrations (0-100 uM). MMPs are secreted as inactive zymogens by the interaction of the zinc ion and the N-terminal (pro) domain. Removal of this domain by a collaborating enzyme initiates protease networks and degradative “cross-talk” to promote remodeling of the ECM (Lopez-Otin, C. et al., Protease degradomics: a new challenge for proteomics. Nat Rev Mol Cell Biol 2002, 3 (7), 509-19 and Overall, C. M. et al., Strategies for MMP inhibition in cancer: innovations for the post-trial era. Nat Rev Cancer 2002, 2 (9), 657-72.) Based on these activity profiles, the loss of MMP3 activity was attributed to the disruption of the protease-web. Cell death at ≤100 uM ilomastat was accessed (<1%) by flow cytometry (propidium iodide stain, data not shown).
The docking of MMP Inhibitors. Using ilomastat as a template several derivatives were modeled as potential inhibitors of MMP-3. Specifically, all ilomastat derivatives contain the same Leu-Trp backbone, but have different ZBGs (Table 1). For each compound, the respective top five scoring poses had very similar conformations. Without being limited thereto, the top scoring conformer is discussed herein.

In the docking of ilomastat, it was found that the top scoring pose had the alkoxide and carbonyl oxygen of the hydroxamic acid functional group ligating the Zn²⁺. The respective calculated distances of 1.913 Å and 1.707 Å for r(C═O . . . Zn²⁺) and r(O⁻. . . Zn²⁺) indicate a strong interaction with the Zn center. This bidentate binding of ilomastat to the Zn²⁺ resulted in the metal center having a trigonal bipyrimidal coordination geometry. Moreover, with the O⁻ atom of the hydroxamic acid ligated to the Zn center Glu202 is able to act as a hydrogen bond donor to the hydroxamic acid alkoxide atom where a ^{Glu202}O . . . O⁻ distance of 2.815 Å was calculated. Lastly, the bidentate ligation of ilomastat to Zn²⁺ allowed the amide group of the hydroxamic acid to hydrogen bond to the backbone carbonyl of Ala165 where r(^{Ala165}C═O . . . N) was calculated to be 2.971 Å. ). This bonding/interaction between hydroxamic acids and MMP3 is discussed in Jacobsen et al., To bind zinc or not to bind zinc: an examination of innovative approaches to improved metalloproteinase inhibition. Biochim Biophys Acta 2010, 1803 (1), 72-94.

Regarding the ligands not containing acidic protons, (i.e., Leu-Trp, AP-1, AP-2, and AP-5), none of the ligands had the proposed ZBGs (Table 1) bind to the Zn²⁺. In the case of Leu-Trp, AP-1, and AP-2, the interaction with the Zn²⁺ ion occurred via carbonyl groups in the Leu-Trp backbone. Specifically, Leu-Trp ligated the Zn²⁺ via the Trp and Leu carbonyl 0-atoms where r(^{Trp}C═O . . . Zn) and r(^{Trp}C═O . . . Zn) were calculated to be 2.092 Å and 2.197 Å, respectively. In the case of AP-1, it was found to only ligate to the Zn²⁺ via the carbonyl oxygen atom of Leu where r(^{Leu}C═O . . . Zn)=2.228 Å. Similarly, for AP-2 it was found to only monodentately ligate to the Zn²⁺, however, it was via the Trp carbonyl 0-atom where r(^{Trp}C═O . . . Zn)=2.171 Å. Regarding AP-5, it was found that an oxygen from the sulfonamide coordinated to the Zn²⁺ ion where r(S═O . . . Zn)=2.006 Å where the amine did not ligate to the Zn²⁺ center. For the top scoring poses of Leu-Trp, AP-1, AP-2, and AP-5 no hydrogen bonding interaction was seen between Glu202 and the ZBG. Moreover, no hydrogen bonding interaction between the backbone carbonyl of Ala165 and the ZBG was observed.

For the ionizable ligands (i.e., AP-3, AP-4, AP-6, and AP-7) all were found to have the ZBG ligate the Zn center. For AP-3, the ZBG was found to monodentately ligate to the Zn²⁺ ion via the anionic thiolate where r(S⁻. . . Zn²⁺) was calculated to be 2.162 Å. However, it was found that the ^{Glu202}CO₂⁻. . . S distance was 4.183 Å. Given the considerable length, it is unlikely that a suitable H-bond interaction would exist between Glu202 and the ZBG of AP-3, thus enhancing the binding of the ligand. Regarding the possible hydrogen bond to Ala165 no H-bonding interaction was observed for AP-3. For AP-4 the sulfate was found to form a bidentate interaction where two of the oxygen atoms coordinated to Zn²⁺. For AP-4 the sulfate binds to the Zn²⁺ in a germinal type binding where the S═O . . . Zn²⁺ distances were calculated to be 2.031 Å and 2.671 Å. The ^{glu202}CO₂⁻. . . O═S distance was calculated to be 2.895 Å indicating the presence of a H-bonding interaction. Regarding the possible hydrogen bond to Ala165 no H-bonding interaction was observed for AP-4.

In the docking of AP-6 and AP-7 it was found that both ligands bidentately ligate to the Zn²⁺. However, unlike ilomastat where the carbonyl and alkoxide of the hydroxamic acid ligates the Zn²⁺, it was found that for AP-6 and AP-7 the bidentate ligation occurs through the O⁻ and N atom of AP-6 and the S⁻ and N atoms of AP-7. Without being bond by theory, this difference between ilomastat and AP-6/AP-7 was likely due to the presence of the sulfonyl functional group that results in the amine not being planar which allows the lone pair to engage in bonding to the Zn²⁺ ion via a side-on interaction. In the case of AP-6, the calculated r(N . . . Zn²⁺) and r(O⁻. . . Zn²⁺) distances were calculated to be 2.017 Å and 1.966 Å, respectively indicating a strong interaction with the Zn²⁺ ion. Concerning Glu202, it was found that r(^{Glu202}CO₂ . . . O⁻)═2.891 Å indicated a strong H-bonding interaction with AP-6. Similarly, for AP-7, a strong interaction with the Zn²⁺ ion existed where the calculated r(N . . . Zn²⁺) and r(S⁻. . . Zn²⁺) distances were 2.124 Å and 2.267 Å, respectively. Regarding Glu202, a weak H-bond interaction existed where r(^{Glu202}CO₂ . . . S) was calculated to be 3.200 Å.

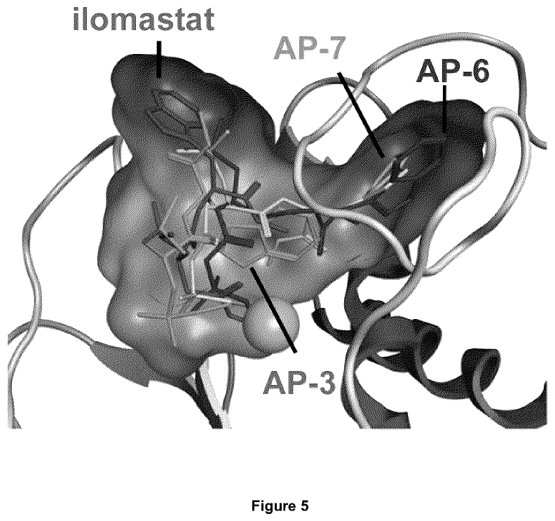

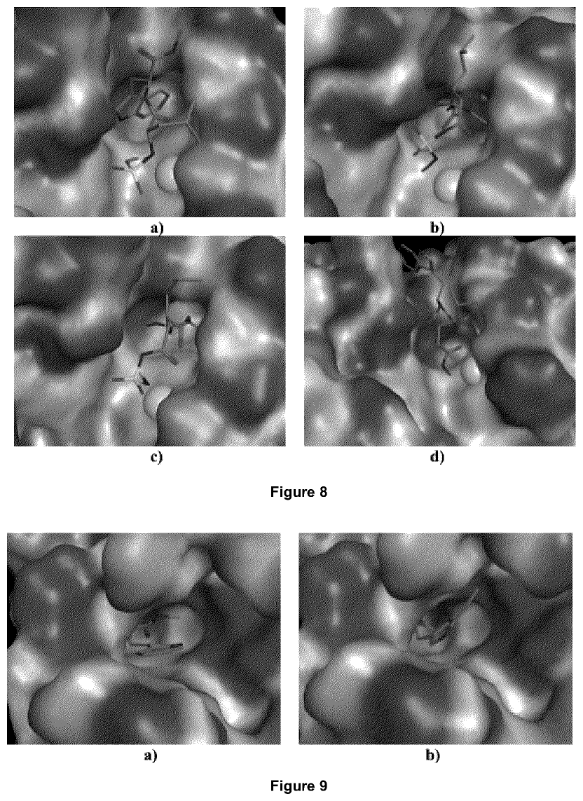

FIG. 5 shows the placement of AP-3, AP-6, AP-7 and ilomastat in the binding site of MMP3. In all cases, the ZBG of each compound is ligated to the Zn²⁺ ion, however the longer ZBG of AP-6 and AP-7 results in a different binding mode between ligand and MMP3 than seen for ilomastat. From FIG. 5 the imidazole functional group of ilomastat is located in a lipophilic region which is a favourable interaction. For AP-6 and AP-7, the leucine side chain is, however, located in this region. For AP-6 and AP-7, the imidazole ring is instead located in a different lipophilic region. In the case of AP-3, it can be seen that neither the imidazole nor the leucine side chain is located in a lipophilic region providing some understanding of its weaker Gibbs binding energy (see below). FIGS. 8 and 9 show another perspective of the binding of AP-3, AP-6, AP-7 and ilomastat to MMP3.

The calculated Gibbs binding energies of the compounds tested are provided in Table 1. From Table 1, it can be seen that in general the presence of an acidic proton resulted in the Gibbs binding energies being more negative than those ligands without acidic protons. The exception being AP-4. Moreover, those ligands that formed a hydrogen bonding interaction with Glu202 resulted in increased Gibb binding energy between MMP3 and ligand (AP-3, AP-6, and AP-7). Such results are in agreement with Jacobsen et al., To bind zinc or not to bind zinc: an examination of innovative approaches to improved metalloproteinase inhibition. Biochim Biophys Acta 2010, 1803 (1), 72-94. From the Gibbs binding energies provided in Table 1, the compound AP-6 was found to have a more negative Gibbs binding energy, therefore, a potentially stronger inhibitor than ilomastat. Without being bound by theory, the bidentate ligation by the anionic binding group contributes to strong binding to the Zn²⁺. Regarding AP-3, even though the ligand only ligates to the Zn²⁺ ion through the S-atom it still has a relatively negative Gibbs binding energy of −11.2 kcal mol⁻¹. Again, without being bond by theory, the lower binding energy of AP-3 is likely due to less favorable placement of the side chains of backbone Trp and Leu residues.

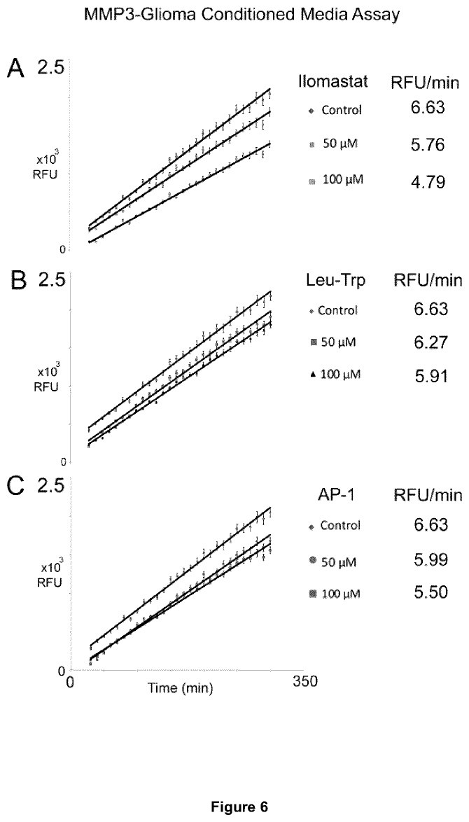

The synthesis of the MMP Inhibitors and NFF3 analysis. The leucine-tryptophan (Leu-Trp) backbone, AP-1, and AP-2 were synthesized. All synthesized compounds were analyzed by mass spectrometry (FIG. 3). The NFF-3 assay was used to determine the biological inhibition of MMP-3 activity for ilomastat and synthesized compounds, Leu-Trp, and AP-1 relative to untreated controls. NFF-3 is a substrate selective for MMP3 (kcat/Km=218,000 s-1 M-1), and to a much lesser degree, MMP9 (kcat/Km=10,000 s-1 M-1) (Nagase, H. et al., Design and characterization of a fluorogenic substrate selectively hydrolyzed by stromelysin 1 (matrix metalloproteinase-3). J Biol Chem 1994, 269 (33), 20952-7). Consistent with our MMP3 SDS-PAGE/westernblot datum, the conditioned medium of C6-Cx43 demonstrated robust fluorescence activity compared to low motility C6 cells and unconditioned DMEM (Aftab, Q. et al., Cx43-Associated Secretome and Interactome Reveal Synergistic Mechanisms for Glioma Migration and MMP3 Activation. Front Neurosci 2019, 13, 143). Linear responses of MMP3 activity was robustly measured from about 30 to about 300 minutes. All compounds were tested at a concentration of 50 μM and 100 μM, and compared to determine MMP3 suppression. From FIG. 6, concentration dependent inhibition was observed. All compounds showed that the 100 μM was better at inhibiting MMP-3 than the 50 μM concentration. This analysis also demonstrated that the Leu-Trp backbone can act as a competitive inhibitor. Consistent with this interpretation, as discussed above in the docking study, Leu-Trp was found to ligate the Zn²⁺ via the Trp and Leu backbone carbonyl O-atoms. Demonstrating the relative contributions of the ZBG in AP-1 and ilomastat, both compounds perform better than Leu-Trp at inhibiting MMP-3.

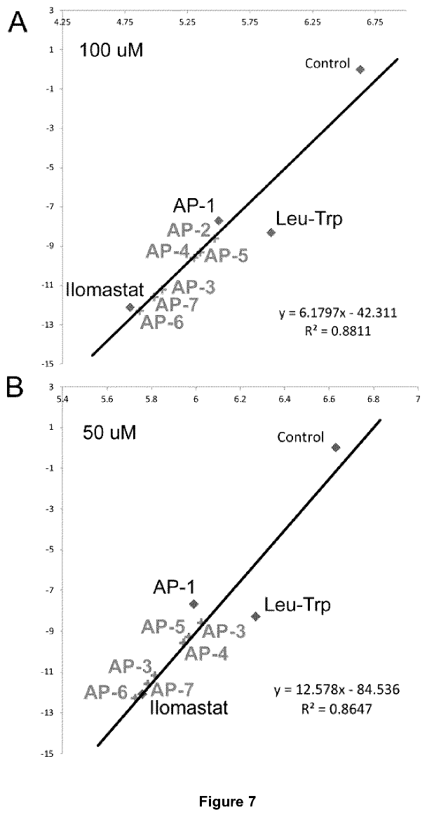

Binding energy scores provided input to predict the performance of the sulfonamide-based MMPIs in biological matrices relevant to invasive GBM. The change in RFU value for each compound was normalized to the control, resulting in a value of less than one. Normalized values were plotted against overall binding energy (FIG. 7). A line of best fit was generated with an R² value of 0.8647 for the inhibitors at 50 μM, and 0.8811 for inhibitors at 100 μM. This indicated a good correlation between the experimental and computational results. Inhibitory potential for the remaining compounds not synthesized are included within the graph (FIGS. 7a and b), based on calculated values using the following relationship:

$$MMP 3 {Activity}_{50 uM} = \frac{Binding Affinity + 84.536}{12.578} \qquad ( eq . 1 )$$

$$MMP 3 {Activity}_{100 uM} = \frac{Binding Affinity + 42.311}{6.1797} \qquad ( eq . 2 )$$

From FIG. 7, although the sulfonamide-based compounds were determined to act as MMPIs, the compounds AP-3, AP-6 and AP-7 were shown to have similar or better biological performance than ilomastat.

Refinement of the binding affinities. Using the top scoring docking pose for ilomastat, AP-3, AP-6, and AP-7, 10 ns MD simulations (see Methods for more detail) were ran to investigate the effect of protein dynamics on the inhibitory effects of ilomastat, AP-3, AP-6, and AP-7. Using the trajectories from the MD simulations, the Gibbs binding energies for each snapshot from the simulation was calculated using the MD_Analysis tool in MOE. Specifically, the binding energy was calculated using the GBVI/WSA dG scoring function. The results of which have been averaged (with standard deviations provided) for ilomastat, AP-3, AP-6, and AP-7 and are provided in Table 2.

TABLE 2
Average lengths for key bonds between ligand and MMP3. Average
Gibbs binding energies with standard deviations.

| | | | ^{Glu202}CO₂ . . . O | Δ_{bind}G |
|---|---|---|---|---|
| Model | O—Zn (Å) | N—Zn (Å) | (Å) | (kcal mol⁻¹) |
| AP-3 | 1.908 ± 0.032 | N/A | 2.990 ± 0.142 | −10.8 ± 0.3 |
| AP-6 | 1.728 ± 0.032 | 1.847 ± 0.040 | 2.645 ± 0.102 | −13.7 ± 0.4 |
| AP-7 | 1.964 ± 0.377^{b} | 1.962 ± 0.071 | 2.921 ± 0.107^{c} | −13.3 ± 0.4 |
| Ilomastat | 1.717 ± 0.030 | 1.966 ± 0.091^{a} | 2.653 ± 0.102 | −13.1 ± 0.4 |

^{a}in the case of ilomastat the coordinating atom was the carbonyl oxygen of the hydroxamic acid functional group and not the nitrogen as seen for AP-6 and AP-7.
^{b}the coordinating atom was the sulfur of AP-7.
^{c}the average distance is for the ^{Glu202}CO₂ . . . S interaction.

From the values in Table 2 it can be seen that in general the binding energies become more negative indicating a stronger binding to MMP3. The AP-3 had a binding energy that was slightly less negative. From the values provided in Table 2, AP-6 and AP-7 are predicted to bind to MMP-3 stronger than ilomastat. Specifically, for ilomastat, Δ_{bind}G=−13.1±0.4 kcal mol⁻¹, whereas for AP-6 and AP-7 Δ_{bind}G was calculated to be −13.7±0.4 kcal mol⁻¹ and 13.4±0.4 kcal mol⁻¹, respectively.

From Table 2, it was shown that ilomastat, AP-3, AP-6, and AP-7 remained strongly ligated to the Zn²⁺ ion. Moreover, from the simulations it can be see that for all four compounds the active site Glu202 interaction has become stronger given the shorter average ^{Glu202}CO₂ . . . O distance than seen in the docking simulation.

Regarding the involvement of Ala165 in the binding of ilomastat, AP-3, AP-6, and AP-7, it was found from the MD simulation for ilomastat ^{Ala165}C═O . . . N=2.925 Å±0.188 Å. For AP-3, the ^{Ala165}C═O . . . N distance was calculated to be 4.384 Å±0.309 Å and for AP-6, the ^{Ala165}C═O . . . N distance was calculated to be 4.384 Å±0.309 Å. In the case of AP-7, the ^{Ala165}C═O . . . N distance was calculated to be 4.343 Å±0.294 Å. From the results, it appeared that the presence of an anionic ZBG that bidentately ligates to the Zn²⁺ ion as well as the ability to form a hydrogen bond to Glu202 provided more potent MMP3 inhibitors whereas the interaction with Ala165 is not predicted to be critical to the inhibition of MMP3. Inhibitors with thiol groups rather than hydroxyl groups were more selective to the zinc containing MMPs. AP-6 and AP-7 are both predicted to bind to MMP3 but the presence of the thiol in AP-7 may enhance the selectivity of AP-7 to the Zn²⁺ containing MMPs versus non-Zn containing metalloproteins.

Patent applications, patents, and publications are cited herein to assist in understanding the embodiments described. All such references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supersede and/or take precedence over any such contradictory material.

Although specific embodiments of the invention have been disclosed herein in detail, it will be understood by those skilled in the art that variations may be made thereto without departing from the spirit of the invention or the scope of the appended claims.

It will be understood that certain of the above-described structures, functions, and operations of the above-described embodiments are not necessary to practice the present invention and are included in the description simply for completeness of an exemplary embodiment or embodiments. In addition, it will be understood that specific structures, functions, and operations set forth in the above-described referenced patents and publications can be practiced in conjunction with the present invention, but they are not essential to its practice. It is therefore to be understood that the invention may be practiced otherwise than as specifically described without actually departing from the spirit and scope of the present invention as defined by the appended claims.

We claim:

1. A compound of Formula I: 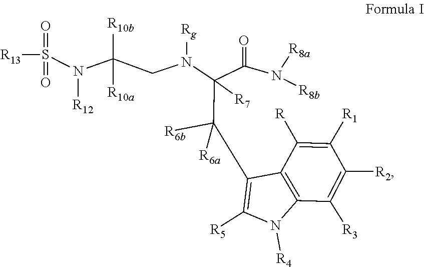
a salt, hydrate, solvate, tautomer, enantiomer, racemate, diastereomer, or combination thereof;
wherein:
R, R₁, R₂, R₃, R₄, R₅, R₆ₐ, R_{6b}, R₇, R₈ₐ, R_{8b}, R₉, R₁₀ₐ, R_{10b}, and R₁₂, are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic; and
R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group,
substituted or unsubstituted aromatic, or a substituted or unsubstituted heteroaromatic.

2. A compound of Formula II: 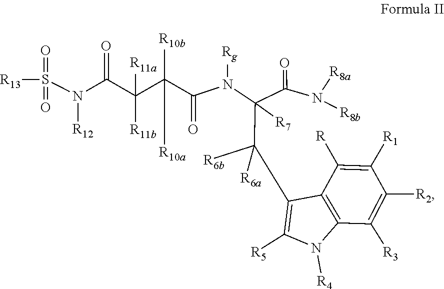
a salt, hydrate, solvate, tautomer, enantiomer, racemate, diastereomer, or combination thereof; wherein:
R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂, are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic; and
R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

3. The compound according to claim 1 or 2, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, a substituted or unsubstituted alkyl group, a substituted or unsubstituted alkenyl group, a substituted or unsubstituted alkynyl group, a substituted or unsubstituted aromatic group, a substituted or unsubstituted heteroaromatic group, a substituted or unsubstituted carbocyclic group, or a substituted or unsubstituted heterocyclic group.

4. The compound according to claim 3, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, a substituted or unsubstituted alkyl group, a substituted or unsubstituted haloalkyl group, a substituted or unsubstituted hydroxyalkyl group, a substituted or unsubstituted cyanoalkyl group, a substituted or unsubstituted alkenyl group, a substituted or unsubstituted C₁-C₆alkylcarbonyl group, a substituted or unsubstituted alkynyl group, a substituted or unsubstituted cycloalkyl group, a substituted or unsubstituted cycloalkenyl group, a substituted or unsubstituted alkylcycloalkyl group, a substituted or unsubstituted alkylcycloalkenyl group, a substituted or unsubstituted heterocycloalkyl group, a substituted or unsubstituted alkylheterocycloalkyl group, a substituted or unsubstituted heterocycloalkenyl group, a substituted or unsubstituted alkylheterocycloalkenyl group, a substituted or unsubstituted aryl group, a substituted or unsubstituted heteroaryl group, a substituted or unsubstituted alkylaryl group, or a substituted or unsubstituted alkylheteroaryl group.

5. The compound according to claim 4, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted alkyl group.

6. The compound according to claim 5, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted C₁-C₆ alkyl group.

7. The compound according to claim 6, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3 dimethyl-2-butyl, 3,3-dimethyl-2-butyl, or hexyl.

8. The compound according to claim 7, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

9. The compound according to claim 8, wherein R, R₁, R₂, R₃, R₄, R₅, R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, or t-butyl.

10. The compound according to claim 1 or 2, wherein R, R₁, R₂, R₃, R₄, and R₅ are each H and R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted alkyl group.

11. The compound according to claim 10, wherein R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted C₁-C₆ alkyl group.

12. The compound according to claim 11, wherein R₆ₐ, R_{6b}, R₇, R₈ₐ, R_{8b}, R₉, R₁₀ₐ, R_{10b}, R₁₁ₐ, R_{11b}, and R₁₂ are each independently selected from H or a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2 methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

13. The compound according to claim 12, wherein R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

14. The compound according to claim 13, wherein R₆ₐ, R_{6b}, R₇, R₈ₐ, R_{8b}, R₉, R₁₀ₐ, R_{10b}, R₁₁ₐ, R_{11b}, and R₁₂ are each independently selected from H, methyl, ethyl, 1-propyl, i-propyl, 1-butyl, i-butyl, sec-butyl, or t-butyl.

15. The compound according to claim 1 or 2, wherein R, R₁, R₂, R₃, R₄, R₅, R₆ₐ, R_{6b}, R₇, R₉, R₁₁ₐ, R_{11b}, and R₁₂ are each H and R₈ₐ, R_{8b}, R₁₀ₐ, and R_{10b} are each independently selected from H or a substituted or unsubstituted alkyl group.

16. The compound according to claim 15, wherein R₈ₐ, R_{8b}, R₁₀ₐ, and R_{10b} are each independently selected from H or a substituted or unsubstituted C₁-C₆ alkyl group.

17. The compound according to claim 16, wherein R₈ₐ, R_{8b}, R₁₀ₐ, and R_{10b} are each independently selected from H or a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

18. The compound according to claim 17, wherein R₈ₐ, R_{8b}, R₁₀ₐ, and R_{10b} are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3 methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

19. The compound according to claim 18, wherein R₈ₐ, R_{8b}, R₁₀ₐ, and R_{10b} are each independently selected from H, methyl, ethyl, 1-propyl, i-propyl, 1-butyl, i-butyl, sec-butyl, or t-butyl.

20. The compound according to claim 1 or 2, wherein R, R₁, R₂, R₃, R₄, R₅, R₆ₐ, R_{6b}, R₇, R₈ₐ, R₉, R₁₀ₐ, R₁₁ₐ, R_{11b}, and R₁₂ are each H and R_{8b} and R_{10b} are each independently selected from H or a substituted or unsubstituted alkyl group.

21. The compound according to claim 20, wherein R_{8b} and R_{10b} are each independently selected from H or a substituted or unsubstituted C₁-C₆ alkyl group.

22. The compound according to claim 21, wherein R_{8b} and R_{10b} are each independently selected from H or a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

23. The compound according to claim 22, wherein R_{8b} and R_{10b} are each independently selected from H, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3 methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

24. The compound according to claim 23, wherein R_{8b} and R_{10b} are each independently selected from H, methyl, ethyl, 1-propyl, i-propyl, 1-butyl, i-butyl, sec-butyl, or t-butyl.

25. The compound according to claim 23, wherein R_{8b} is selected from methyl or ethyl, and R_{10b} is selected from methyl, ethyl, 1-propyl, i-propyl, 1-butyl, i-butyl, sec-butyl, or t-butyl.

26. The compound according to claim 23, wherein R_{8b} is methyl and R_{10b} is selected from 1-butyl, i-butyl, sec-butyl, or t-butyl.

27. The compound according to claim 23, wherein R_{8b} is selected from methyl, and R_{10b} is i-butyl.

28. The compound according to any one of claims 1 to 27, wherein R₁₃ is selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

29. The compound according to claim 28, wherein R₁₃ is selected from H, hydroxyl group, a substituted or unsubstituted thiol group, a substituted or unsubstituted amino group, a substituted or unsubstituted alkyl group, a substituted or unsubstituted alkenyl group, a substituted or unsubstituted alkynyl group, a substituted or unsubstituted aromatic group, a substituted or unsubstituted heteroaromatic group, a substituted or unsubstituted carbocyclic group, or a substituted or unsubstituted heterocyclic group.

30. The compound according to claim 29, wherein R₁₃ is selected from H, hydroxyl group, a substituted or unsubstituted thiol group, a substituted or unsubstituted amino group, a substituted or unsubstituted alkyl group, a substituted or unsubstituted haloalkyl group, a substituted or unsubstituted hydroxyalkyl group, a substituted or unsubstituted aminoalkyl group, a substituted or unsubstituted thioalkyl group, a substituted or unsubstituted alkylamino group, a substituted or unsubstituted alkylthio group, a substituted or unsubstituted alkoxy group, a substituted or unsubstituted carboxy group, a substituted or unsubstituted carbonyl group, a substituted or unsubstituted alkenyl group, a substituted or unsubstituted alkynyl group, a substituted or unsubstituted cyanoalkyl, a substituted or unsubstituted alkenyl, a substituted or unsubstituted C₁-C₆ alkylcarbonyl, a substituted or unsubstituted alkynyl, a substituted or unsubstituted cycloalkyl, a substituted or unsubstituted cycloalkenyl, a substituted or unsubstituted alkylcycloalkyl, a substituted or unsubstituted alkylcycloalkenyl, a substituted or unsubstituted heterocycloalkyl, a substituted or unsubstituted alkylheterocycloalkyl, a substituted or unsubstituted heterocycloalkenyl, a substituted or unsubstituted alkylheterocycloalkenyl, a substituted or unsubstituted aryl, or a substituted or unsubstituted heteroaryl.

31. The compound according to claim 28, wherein R₁₃ is selected from H, hydroxyl group, substituted or unsubstituted alkyl group, substituted or unsubstituted alkenyl group, substituted or unsubstituted alkynyl group, —NR₁₄R₁₅, —CR₁₆R₁₇R₁₈, —C(O)NR₁₉R₂₀, halo group, —S(O)R₂₁, —SO₂R₂₂, —R₂₃S(O)R₂₄, —R₂₅SO₂R₂₆, —R₂₇SR₂₈, —R₂₇S—C(O)R₂₈, —SR₂₉, —S—C(O)R₃₀, —C(O)SR₃₁, —N(R₃₂)C(O)R₃₃, —C(O)R₃₄, —C(O)OR₃₅, or —OR₃₆, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

32. The compound according to claim 31, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group, a substituted or unsubstituted haloalkyl group, a substituted or unsubstituted hydroxyalkyl group, a substituted or unsubstituted cyanoalkyl group, a substituted or unsubstituted alkenyl group, a substituted or unsubstituted C₁-C₆alkylcarbonyl group, a substituted or unsubstituted alkynyl group, a substituted or unsubstituted cycloalkyl group, a substituted or unsubstituted cycloalkenyl group, a substituted or unsubstituted alkylcycloalkyl group, a substituted or unsubstituted alkylcycloalkenyl group, a substituted or unsubstituted heterocycloalkyl group, a substituted or unsubstituted alkylheterocycloalkyl group, a substituted or unsubstituted heterocycloalkenyl group, a substituted or unsubstituted alkylheterocycloalkenyl group, a substituted or unsubstituted aryl group, or a substituted or unsubstituted heteroaryl group.

33. The compound according to claim 32, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group.

34. The compound according to claim 33, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, or a substituted or unsubstituted C₁-C₆ alkyl group.

35. The compound according to claim 34, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2 methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

36. The compound according to claim 35, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

37. The compound according to claim 36, wherein R₁₄, R₁₅, R₁₆R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, or t-butyl.

38. The compound according to claim 28, wherein R₁₃ is selected from —NR₁₄R₁₅, —CR₁₆R₁₇R₁₈, —R₂₇SR₂₈, —R₂₇S—C(O)R₂₈, —C(O)SR₃₁, —N(R₃₂)C(O)R₃₃, —C (O)R₃₄, —C(O)OR₃₅, or —OR₃₆, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, carboxylic acid group, phosphate group, halo group, hydroxyl group, a substituted or unsubstituted thiol group, a substituted or unsubstituted amino group, nitro group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

39. The compound according to claim 38, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group, a substituted or unsubstituted haloalkyl group, a substituted or unsubstituted hydroxyalkyl group, a substituted or unsubstituted cyanoalkyl group, a substituted or unsubstituted alkenyl group, a substituted or unsubstituted C₁-C₆alkylcarbonyl group, a substituted or unsubstituted alkynyl group, a substituted or unsubstituted cycloalkyl group, a substituted or unsubstituted cycloalkenyl group, a substituted or unsubstituted alkylcycloalkyl group, a substituted or unsubstituted alkylcycloalkenyl group, a substituted or unsubstituted heterocycloalkyl group, a substituted or unsubstituted alkylheterocycloalkyl group, a substituted or unsubstituted heterocycloalkenyl group, a substituted or unsubstituted alkylheterocycloalkenyl group, a substituted or unsubstituted aryl group, or a substituted or unsubstituted heteroaryl group.

40. The compound according to claim 39, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted alkyl group.

41. The compound according to claim 40, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, or a substituted or unsubstituted C₁-C₆ alkyl group.

42. The compound according to claim 41, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a substituted or unsubstituted thiol group, a substituted or unsubstituted amino group, a substituted or unsubstituted methyl, ethyl, 1-propyl, i propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

43. The compound according to claim 42, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, t-butyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2 methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, or hexyl.

44. The compound according to claim 43, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a thiol group, an amino group, methyl, ethyl, 1-propyl, i-propyl, 1-butyl, i-butyl, sec-butyl, or t-butyl.

45. The compound according to any one of claims 38 to 44, wherein R₁₃ is selected from hydroxyl group, —NR₁₄R₁₅, or —CR₁₆R₁₇R₁₈, wherein R₁₄, R₁₅, R₁₆R₁₇, and R₁₈ are each independently selected from H, halo group, hydroxyl group, a substituted or unsubstituted thiol group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

46. The compound according to claim 45, wherein R₁₄, R₁₅, R₁₆R₁₇, R₁₈, R₂₇, R₂₈, R₃₁, R₃₂, R₃₃, R₃₄, R₃₅, and R₃₆ are each independently selected from H, halo group, hydroxyl group, a thiol group, an amino group, a substituted or unsubstituted alkyl group, or —S—C(O)R₃₇, wherein R₃₇ is selected from H or a substituted or unsubstituted alkyl group.

47. The compound according to claim 45 or 46, wherein R₁₃ is a hydroxyl group.

48. The compound according to claim 45 or 46, wherein R₁₃ is —NR₁₄R₁₅, wherein R₁₄ and R₁₅ are each independently selected from H, halo group, hydroxyl group, a substituted or unsubstituted thiol group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

49. The compound according to claim 48, wherein R₁₄ and R₁₅ are each independently selected from H, halo group, hydroxyl group, a substituted or unsubstituted thiol group, a substituted or unsubstituted amino group, or a substituted or unsubstituted alkyl group.

50. The compound according to claim 48, wherein R₁₄ and R₁₅ are each independently selected from H, hydroxyl group, a thiol group, or a substituted or unsubstituted C₁-C₆ alkyl group.

51. The compound according to claim 48, wherein R₁₄ is H or C₁-C₆ alkyl group and R₁₅ is selected from H, hydroxyl group, or a thiol group.

52. The compound according to claim 45 or 46, wherein R₁₃ is —CR₁₆R₁₇R₁₈, wherein R₁₆, R₁₇, and R₁₈ are each independently selected from H, halo group, hydroxyl group, a substituted or unsubstituted thiol group, a substituted or unsubstituted amino group, a substituted or unsubstituted hydrocarbon group, a substituted or unsubstituted heterogeneous group, a substituted or unsubstituted carbocyclic group, a substituted or unsubstituted heterocyclic group, substituted or unsubstituted aromatic, or a substituted or unsubstituted heteroaromatic.

53. The compound according to claim 52, wherein R₁₆, R₁₇, and R₁₈ are each independently selected from H, halo group, hydroxyl group, a thiol group, an amino group, a substituted or unsubstituted alkyl group, or —S—C(O)R₃₇, wherein R₃₇ is selected from H or a substituted or unsubstituted alkyl group.

54. The compound according to claim 53, wherein R₁₆ and R₁₇ are each independently selected from H or a substituted or unsubstituted alkyl group and R₁₈ is selected from a halo group, hydroxyl group, a thiol group, an amino group, a substituted or unsubstituted alkyl group, or —S—C(O)R₃₇, wherein R₃₇ is selected from H or a substituted or unsubstituted alkyl group.

55. The compound according to claim 53, wherein R₁₆ and R₁₇ are each independently selected from H or a substituted or unsubstituted C₁-C₆ alkyl group and R₁₈ is selected from a halo group, hydroxyl group, a thiol group, an amino group, a substituted or unsubstituted C₁-C₆ alkyl group, or —S—C(O)R₃₇, wherein R₃₇ is selected from H or a substituted or unsubstituted C₁-C₆ alkyl group.

56. The compound according to claim 53, wherein R₁₆ and R₁₇ are each independently selected from H and R₁₈ is selected from a halo group, hydroxyl group, a thiol group, or —S—C(O)R₃₇, wherein R₃₇ is selected from H or a substituted or unsubstituted C₁-C₆ alkyl group.

57. The compound according to claim any one of claims 1 to 56, wherein the compound of Formula I includes at least one compound selected from: 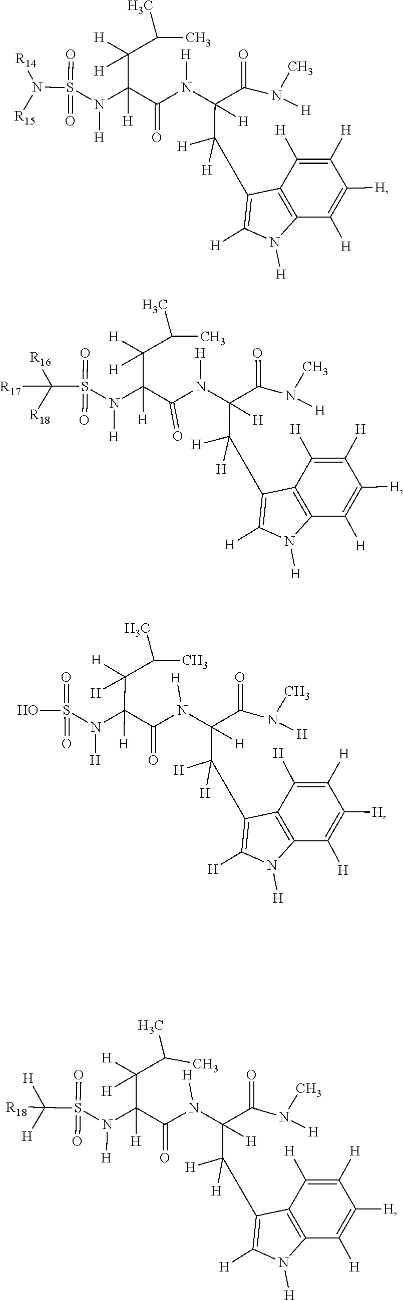
a salt, hydrate, solvate, tautomer, enantiomer, racemate, diastereomer, or combination thereof.

58. The compound according to any one of claims 1 to 57, wherein the compound of Formula I includes at least one compound selected from: 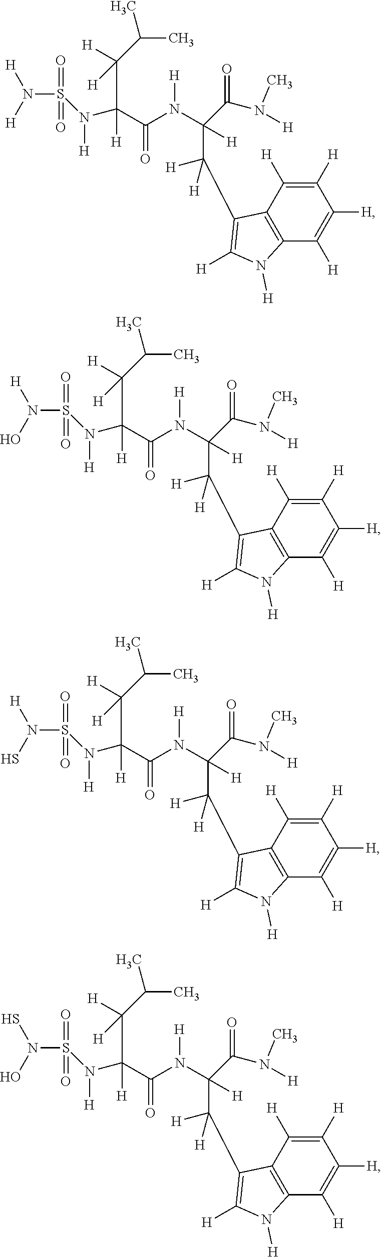 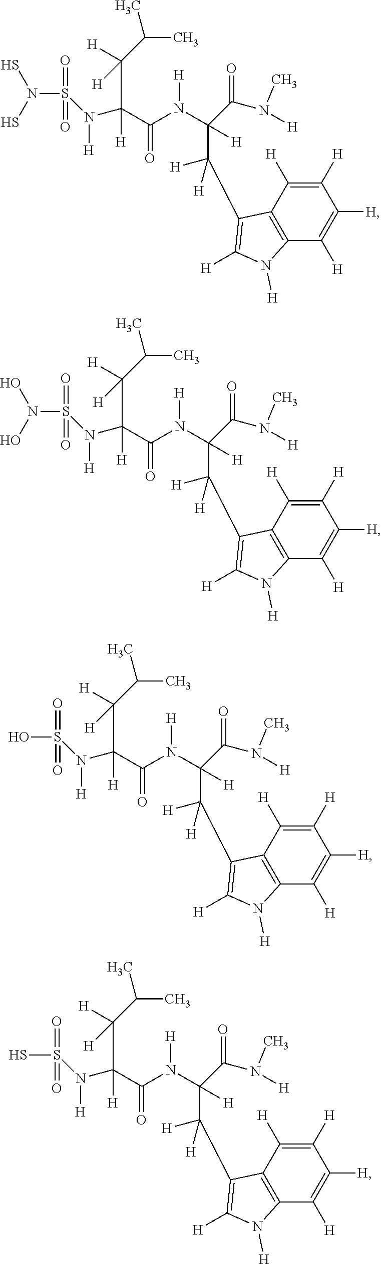 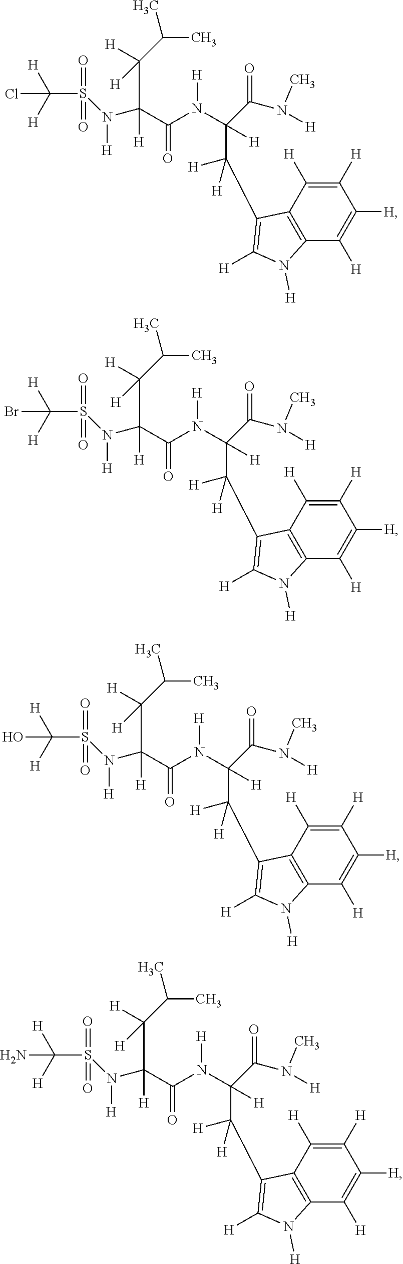 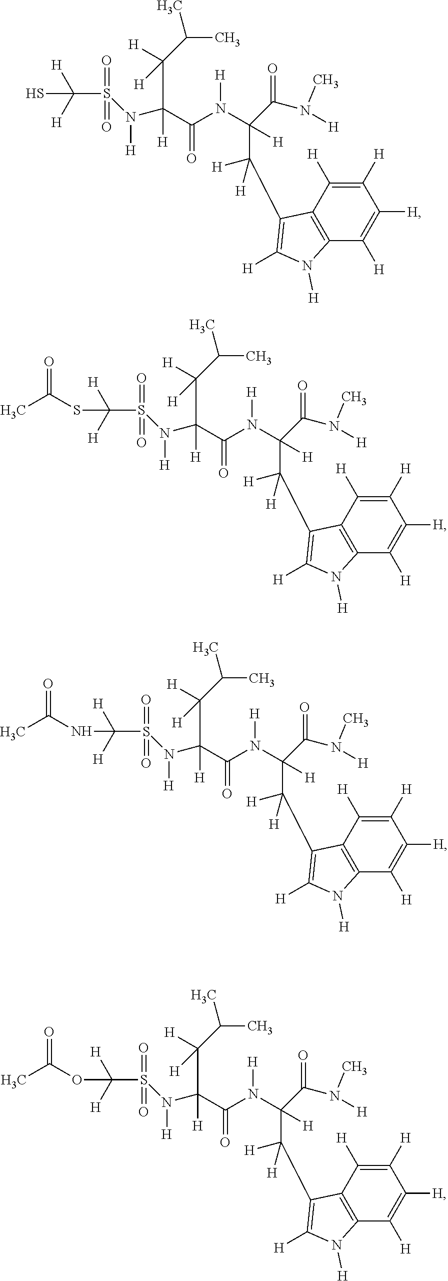 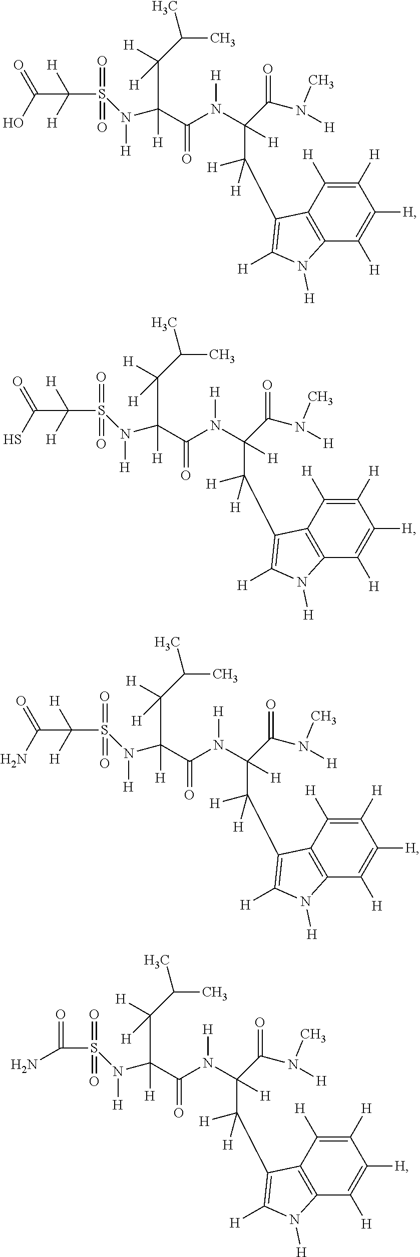 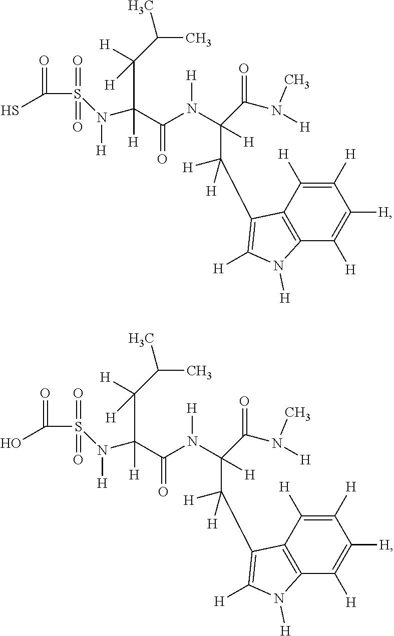
a salt, hydrate, solvate, tautomer, enantiomer, racemate, diastereomer, or combination thereof.

59. The compound according to claim any one of claims 1 to 57, wherein the compound of Formula II includes at least one compound selected from: 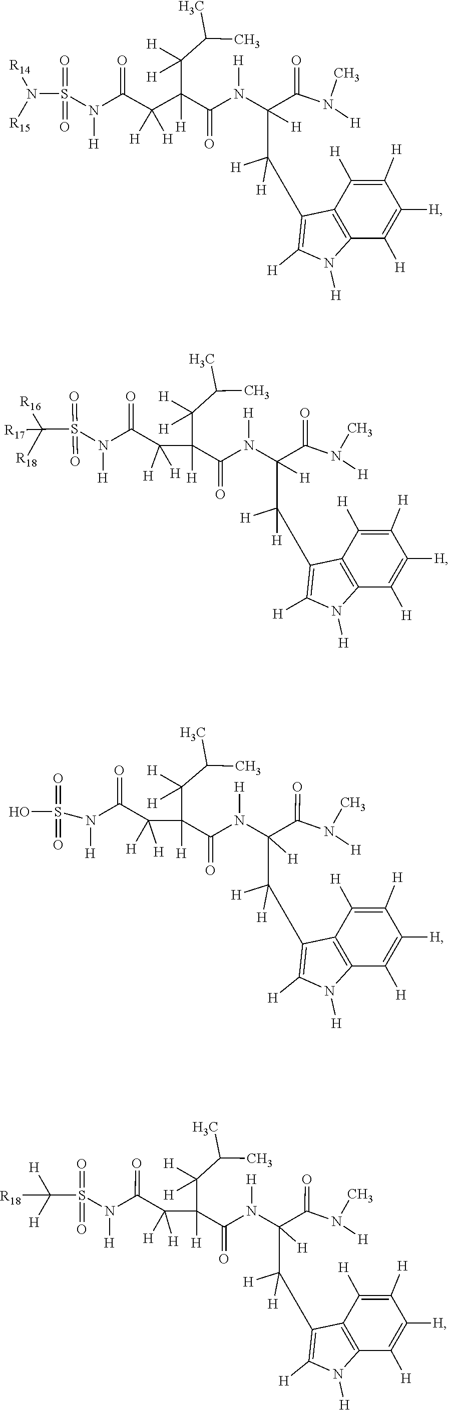
a salt, hydrate, solvate, tautomer, enantiomer, racemate, diastereomer, or combination thereof.

60. The compound according to any one of claims 1 to 57, wherein the compound of Formula II includes at least one compound selected from: 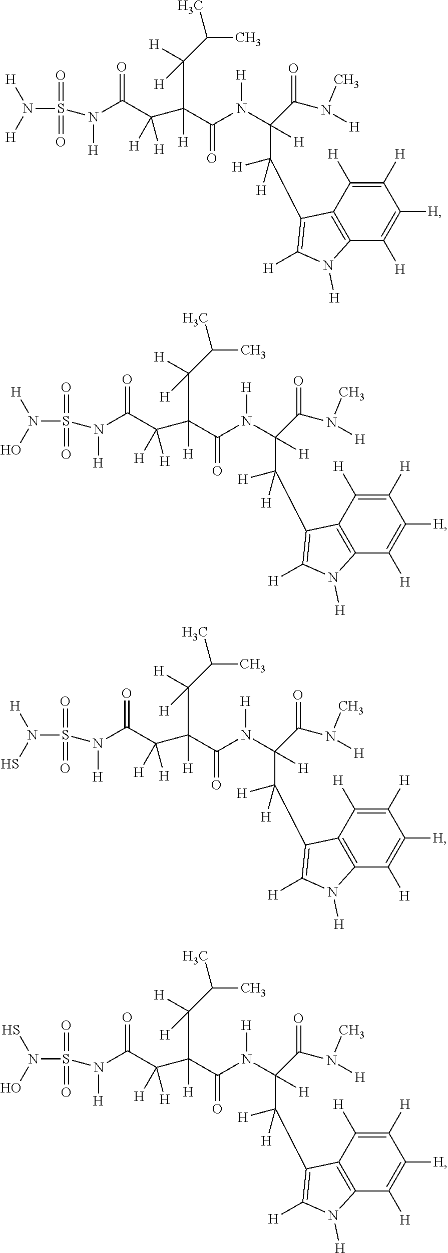 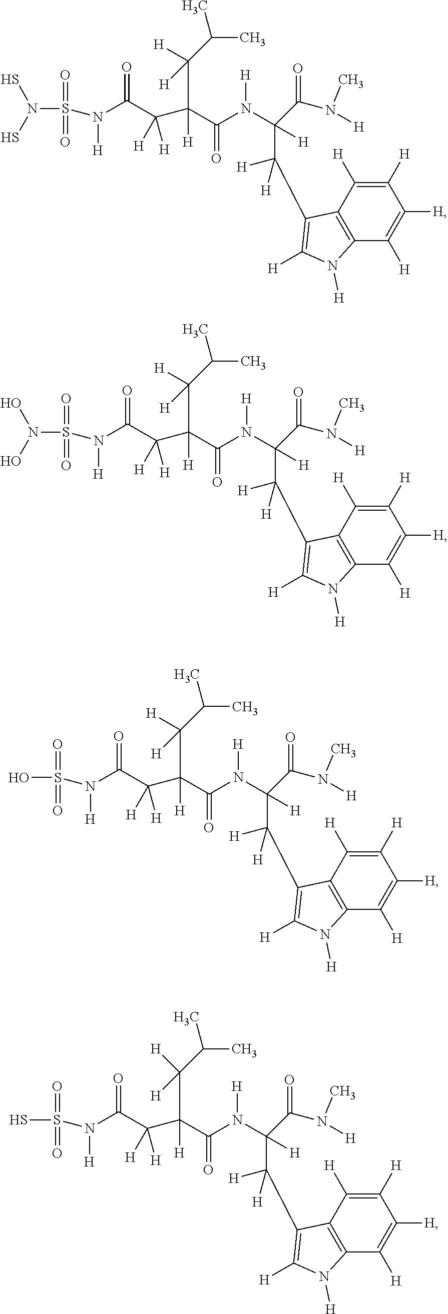 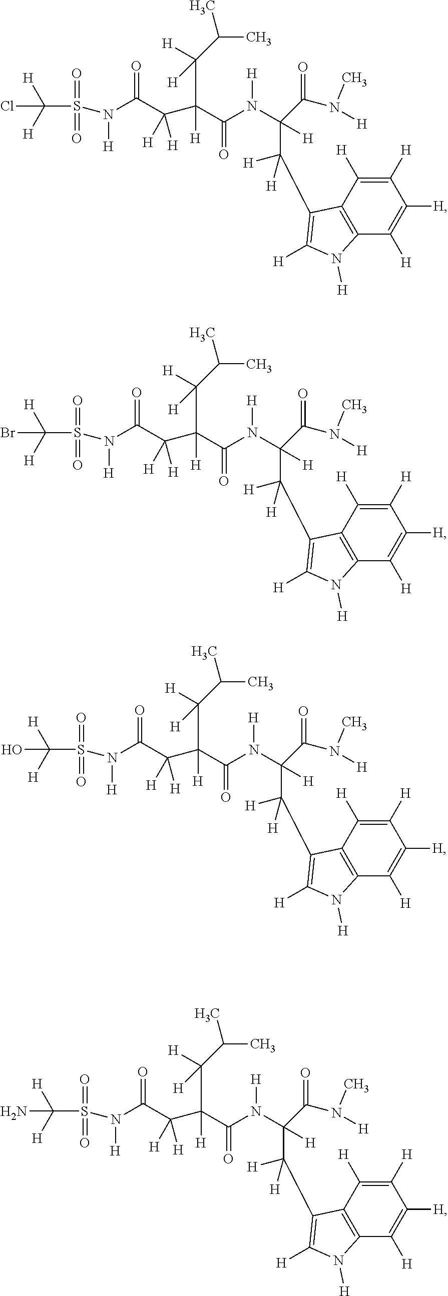 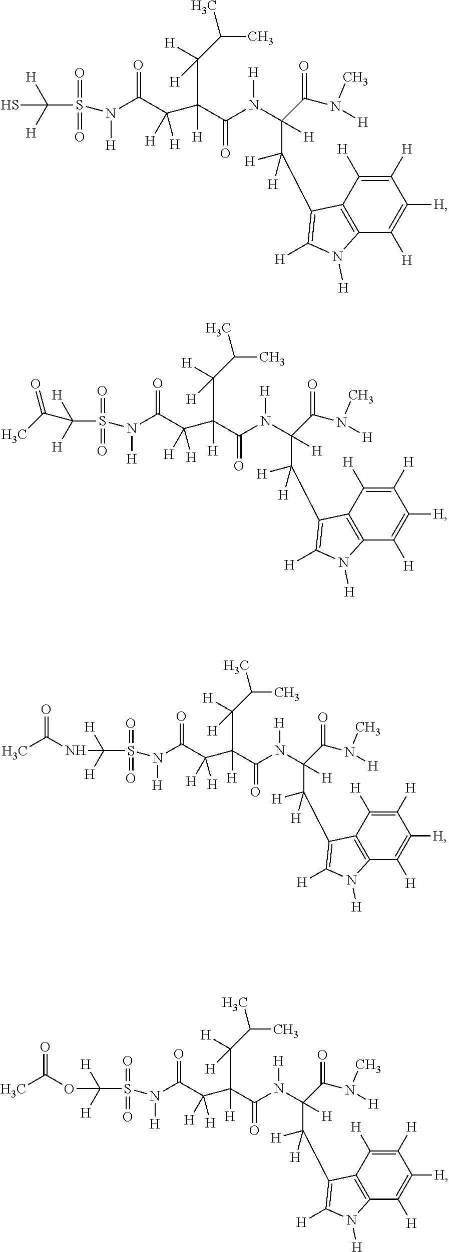 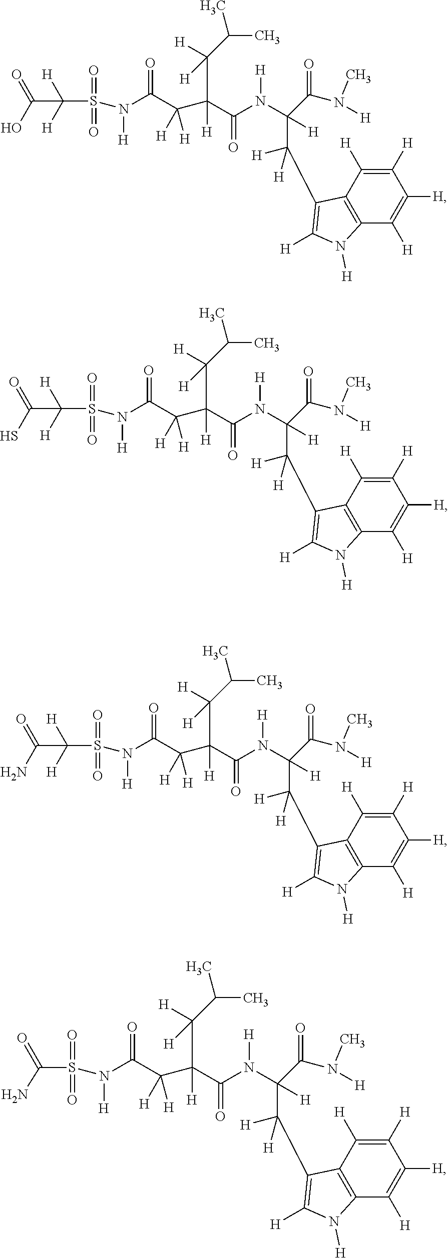 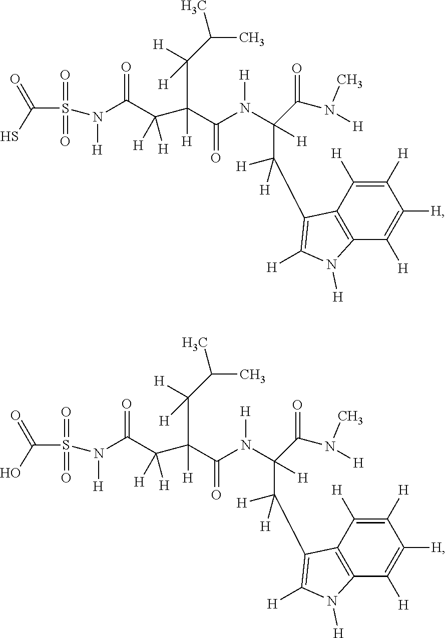
a salt, hydrate, solvate, tautomer, enantiomer, racemate, diastereomer, or combination thereof.

61. The compound according to any one of claims 1 to 57, wherein the compound has an S configuration at the α-carbon with R₇ and an S configuration at the α-carbon with R₁₀ₐ and R_{10b}.

62. The compound according to any one of claims 1 to 61, wherein the compound is a matrix metalloproteinase (MMP) inhibitor.

63. The compound according to claim 62, wherein the matrix metalloproteinase is MMP-3, MMP-8, and/or MMP-13.

64. The compound according to claim 62, wherein the matrix metalloproteinase is MMP-3.

65. The compound according to any one of claims 62 to 64, wherein R₁₃—SO₂NR₁₂— of Formula I and/or Formula II bidentately ligates to the Zn²⁺ ion of a matrix metalloproteinase (MMP) and/or is capable of forming a hydrogen bond to Glu202 of the matrix metalloproteinase (MMP).

66. The compound according to any one of claims 1 to 65 for the treatment of a matrix metalloproteinase mediated condition.

67. The compound according to claim 66, wherein the matrix metalloproteinase mediated condition is selected from cancer, angiogenesis, cardiovascular disease, neurological disease, inflammation, eye disease, autoimmune disease, for regulating contraception, or other conditions that are affected by the regulation of MMPs.

68. The compound of claim 67, wherein the cancer is selected from pancreatic cancer, gastric cancer, lung cancer, colorectal cancer, prostate cancer, cervical cancer, ovarian cancer, cancer of CNS, renal cell cancer, basal cell cancer, breast cancer, bone cancer, brain cancer, lymphoma, leukemia, melanoma, myeloma, leukemia, or other hematological cancers.

69. The compound according to claim 68, wherein the cancer is selected from brain cancer, breast cancer, acute leukemia, chronic leukemia, colorectal cancer, or lung cancer.

70. The compound according to claim 68 or 69, wherein the cancer is GBM.

71. The compound according to any one of claims 68 to 70, wherein the cancer is a carcinoma.

72. The compound according to claim 71, wherein the carcinoma is selected from small cell carcinomas, cervical carcinomas, glioma, astrocytoma, prostate carcinomas, ovarian carcinomas, melanoma, breast carcinomas, brain carcinomas, or colorectal carcinomas.

73. The compound according to any one of claims 1 to 72 in combination with radiation therapy.

74. A pharmaceutical composition comprising the compound according to any one of claims 1 to 73 and at least one pharmaceutically acceptable carrier and/or diluent.

75. The composition according to claim 74 further comprising an anti-cancer agent.

76. A method for the treatment of a matrix metalloproteinase mediated condition in a mammal, comprising administering to the mammal a therapeutically effective amount of the compound according to any one of claims 1 to 65 or the composition of claim 74 or 75.

77. The method according to claim 76, wherein the matrix metalloproteinase mediated condition is selected from cancer, angiogenesis, cardiovascular disease, neurological disease, inflammation, eye disease, autoimmune disease, for regulating contraception, or other conditions that are affected by the regulation of MMPs.

78. The method of claim 77, wherein the cancer is selected from pancreatic cancer, gastric cancer, lung cancer, colorectal cancer, prostate cancer, cervical cancer, ovarian cancer, cancer of CNS, renal cell cancer, basal cell cancer, breast cancer, bone cancer, brain cancer, lymphoma, leukemia, melanoma, myeloma, leukemia, or other hematological cancers.

79. The method according to claim 78, wherein the cancer is selected from brain cancer, breast cancer, acute leukemia, chronic leukemia, colorectal cancer, or lung cancer.

80. The method according to claim 78 or 79, wherein the cancer is GBM.

81. The method according to any one of claims 78 to 80, wherein the cancer is a carcinoma.

82. The method according to claim 81, wherein the carcinoma is selected from small cell carcinomas, cervical carcinomas, glioma, astrocytoma, prostate carcinomas, ovarian carcinomas, melanoma, breast carcinomas, brain carcinomas, or colorectal carcinomas.

83. The method according to any one of claims 76 to 82 in combination with radiation therapy.

84. The method according to any one of claims 76 to 83, wherein the compound or composition is administered orally and/or parenterally.

85. The method according to any one of claims 76 to 84, wherein the mammal is a human.

86. Use of a therapeutically effective amount of the compound according to any one of claims 1 to 65 or the composition of claim 74 or 75 for the treatment of a matrix metalloproteinase mediated condition in a mammal.

87. The use according to claim 86, wherein the matrix metalloproteinase mediated condition is selected from cancer, angiogenesis, cardiovascular disease, neurological disease, inflammation, eye disease, autoimmune disease, for regulating contraception, or other conditions that are affected by the regulation of MMPs.

88. The use of claim 87, wherein the cancer is selected from pancreatic cancer, gastric cancer, lung cancer, colorectal cancer, prostate cancer, cervical cancer, ovarian cancer, cancer of CNS, renal cell cancer, basal cell cancer, breast cancer, bone cancer, brain cancer, lymphoma, leukemia, melanoma, myeloma, leukemia, or other hematological cancers.

89. The use according to claim 88, wherein the cancer is selected from brain cancer, breast cancer, acute leukemia, chronic leukemia, colorectal cancer, or lung cancer.

90. The use according to claim 88 or 89, wherein the cancer is GBM.

91. The use according to any one of claims 88 to 90, wherein the cancer is a carcinoma.

92. The use according to claim 91, wherein the carcinoma is selected from small cell carcinomas, cervical carcinomas, glioma, astrocytoma, prostate carcinomas, ovarian carcinomas, melanoma, breast carcinomas, brain carcinomas, or colorectal carcinomas.

93. The use according to any one of claims 86 to 92 in combination with radiation therapy.

94. The use according to any one of claims 86 to 93, wherein the compound or composition is administered orally and/or parenterally.

95. The use according to any one of claims 86 to 94, wherein the mammal is a human.
